(19) United States
(12) Patent Application Publication
(10) Pub. No.: US 2010/0086540 A1
(43) Pub. Date: Apr. 8, 2010

(54) CO-CRYSTAL OF ANTIBODY 11F8FAB FRAGMENT AND EGFR EXTRACELLULAR DOMAIN AND USES THEREOF

(75) Inventors: PAUL H. KUSSIE, NEW YORK, NY (US); KATHRYN M. FERGUSON, MEDIA, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania and ImClone, LLC

(21) Appl. No.: 12/495,435

(22) Filed: Jun. 30, 2009

Related U.S. Application Data
(63) Continuation of application No. 12/313,663, filed on Nov. 20, 2008, now abandoned.
(60) Provisional application No. 61/003,883, filed on Nov. 20, 2007.

(51) Int. Cl. A61K 39/395; C07K 16/00; C07K 1/00; G01N 33/68; G01N 33/53; A61P 35/00; G06F 19/00

(52) U.S. Cl. 424/130.1; 530/391.1; 530/402; 436/86; 436/501; 530/387.1; 702/19

(57) ABSTRACT

The present invention relates to co-crystals of antibody 11F8 Fab fragments and the complete extracellular domain of EGFR or isolated domain III of EGFR, and structural coordinates obtained from such crystals. Such coordinates are useful for identifying mimetics that bind to the extracellular domain of EGFR. Such mimetics may, for example, inhibit binding of ligands to EGFR, inhibit activation of EGFR, and/or reduce proliferation of tumor cells.

### CROSS REFERENCE TO RELATED APPLICATIONS

The present application claims priority benefit of U.S. Provisional Appl. No. 61/003,883, filed Nov. 20, 2007, which is incorporated by reference herein in its entirety.

### STATEMENT OF FEDERALLY SPONSORED RESEARCH

The work described herein was partially supported by National Cancer Institute grant no. R01-CA112552.

### MEGATABLES

Tables 2 and 3 of this application are lengthy tables and are provided on a CD-ROM, which is incorporated by reference herein in its entirety.

### FIELD OF THE INVENTION

The present invention relates to co-crystals of Fab fragments of the EGFR-binding humanized monoclonal antibody 11F8 in complex with the extracellular domain of EGFR, and structural coordinates obtained from such crystal. Such coordinates are useful for identifying mimetics, preferably EGFR antagonists, that bind to the extracellular domain of EGFR. Such mimetics may for example inhibit binding of ligand to EGFR, inhibit activation of EGFR, and/or reduce proliferation of tumor cells.

### BACKGROUND OF THE INVENTION

Although normal cells proliferate by the highly controlled activation of growth factor receptor tyrosine kinases (“RTKs”) by their respective ligands, cancer cells also proliferate by the activation of growth factor receptors, but lose the careful control of normal proliferation. The loss of control may be caused by numerous factors, such as the overexpression of growth factors and/or receptors, and autonomous activation of biochemical pathways regulated by growth factors. Some examples of RTKs involved in tumorigenesis are the receptors for epidermal growth factor receptor (EGFR) (also known as human EGF receptor-1 (HER1)), platelet-derived growth factor (PDGFR), insulin-like growth factor (IGFR), nerve growth factor (NGFR), and fibroblast growth factor (FGF). Binding of growth factors to these cell surface receptors induces receptor activation, which initiates and modifies signal transduction pathways and leads to cell proliferation and differentiation.

Generally, RTKs have an extracellular region, a transmembrane hydrophobic region, and an intracellular region bearing a kinase domain. The first step in the activation of an RTK is ligand-induced dimerization leading to exposure of phosphorylation sites, activation of the intracellular kinase domain and recruitment of down-stream signaling molecules. The most commonly observed mode of RTK dimerization involves the “crosslinking” of two receptors having exposed dimerization interfaces by binding of a bivalent ligand. For EGFR, structural data published in recent years have led to the proposal of quite a different mechanism. In the absence of ligand, a distinct configuration of the receptor monomer occludes the dimerization interface of the receptor by burying it in an intramolecular “tether.” Ligand binding induces a conformational change in EGFR that exposes this dimerization site, promoting dimerization and receptor activation.

EGFR is a 170 kD membrane-spanning glycoprotein with an extracellular ligand binding domain, a transmembrane region and a cytoplasmic protein tyrosine kinase domain. Examples of ligands that stimulate EGFR include epidermal growth factor (EGF), transforming growth factor-α (TGF-α), heparin-binding growth factor (HBGF), β-cellulin, and Cripto-1. Binding of specific ligands results in EGFR autophosphorylation, activation of the receptor's cytoplasmic tyrosine kinase domain and initiation of multiple signal transduction pathways that regulate tumor growth and survival.

Growth factors that activate EGFR are also thought to play a role in tumor angiogenesis. Angiogenesis, which refers to the formation of capillaries from pre-existing vessels in the embryo and adult organism, is known to be a key element in tumor growth, survival and metastasis. It has been reported that EGFR mediated stimulation of tumor cells leads to increased expression of the angiogenic factors vascular endothelial growth factor (VEGF), interleukin-8 (IL-8), and basic fibroblast growth factor (bFGF), which can lead to activation of tumor-associated vascular endothelial cells. Stimulation of tumor associated vascular endothelial cells may also occur through activation of their own EGF receptors, by tumor produced growth factors such as TGF-α and EGF.

It has been reported that many human tumors express or overexpress EGFR. Expression of EGFR is correlated with poor prognosis, decreased survival, and/or increased metastasis. EGFR, because of this involvement in tumorigenesis, has been specifically targeted for anticancer therapies. These therapies have predominantly included either a monoclonal antibody that blocks binding of ligand to the extracellular domain of the receptor or a synthetic tyrosine kinase inhibitor that acts directly on the intracellular region to prevent signal transduction.

Cetuximab mAb (ERBITUX®) is a recombinant, human/mouse chimeric, monoclonal antibody composed of the Fv regions of a murine anti-EGFR antibody with human IgG1 heavy and kappa light chain constant regions and has an approximate molecular weight of 152 kDa. Cetuximab binds specifically to the extracellular domain of the human EGFR, and is an EGFR antagonist, which blocks ligand binding to EGFR, prevents receptor activation, and inhibits growth of tumor cells that express EGFR. Cetuximab has been approved for use in combination with or without irinotecan in the treatment of patients with epidermal growth factor receptor-expressing, metastatic colorectal cancer who are refractory or can not tolerate irinotecan-based chemotherapy. Cetuximab has been shown to be effective for treatment of psoriasis.

This structural model for ligand-induced EGFR dimerization suggests several possible approaches for inhibition, some of which are exploited by therapeutic antibodies that have emerged from early screens (Ferguson, 2004). For example, the chimeric cetuximab antibody inhibits EGFR activation by competing directly with EGF for its binding site on domain III of the receptor (Li et al., 2005). Cetuximab binding also sterically impedes adoption of the extended configuration. On the other hand, the anti-ErbB2 antibody pertuzumab binds directly to the presumed domain II (hetero)dimerization site of ErbB2 (Franklin et al., 2004), and the anti-EGFR antibody mAb806 binds to a domain II epitope close to the receptor's dimerization site (Johns et al., 2004).

The crystal structure of an EGF-EGFR extracellular domain complex, wherein the receptor domain exists in dimeric form, has been provided Ogiso, H. et al., 2002, Cell 110, 775-787. The structure of an EGF-EGFR extracellular domain complex obtained by crystallization at low, non-physiological pH, wherein the receptor exists in monomeric form has also been provided Ferguson, K. M. et al., 2003, Mol Cell 11, 507-517. The structure of a transforming growth factor alpha (TGF-α)-EGFR extracellular domain complex in dimeric form has also been determined (Garrett, T. P. et al., 2002, Cell 110, 763-773).

Antibody 11F8 is a fully human anti-EGFR extracellular domain antibody isolated from a human Fab phage display library and is disclosed in U.S. Publication No. 2007/0264253 A1, which is incorporated by reference herein in its entirety. Since the variable domains of antibody 11F8 are human, the antigen-binding domains of this antibody do not provoke any hypersensitivity reactions in humans.

The crystal structure of EGFR with cetuximab Fab was previously determined and is disclosed in WO 2006/009694, which is incorporated by reference in its entirety. Cetuximab is a chimeric protein with mouse variable region amino acids fused to human constant region amino acids. The invention disclosed herein provides crystals and atomic coordinates of complexes of the Fab fragment of fully human antibody 11F8 and each of the complete extracellular domain of EGFR and isolated domain III of EGFR. Accordingly, the present invention provides methods for identifying potential mimetics by screening against at least a subset of the coordinates obtained from such a crystal. Mimetics may be assayed for biological activities to obtain EGFR antagonists useful for treatment of EGFR dependent conditions or diseases. EGFR antagonists interact with the receptor to inhibit EGFR tyrosine kinase activity, without limitation, by blocking ligand binding, inhibiting receptor dimerization, ultimately inhibiting receptor substrate phosphorylation, gene activation, and cellular proliferation. Preferably, the antagonists have substantially similar or improved effectiveness as compared to cetuximab and/or antibody 11F8. The antagonists may be used for the treatment of conditions associated with EGFR expression. Such diseases include tumors that express, or overexpress EGFR and which may be stimulated by a ligand of EGFR as well as hyperproliferative diseases stimulated by a ligand of EGFR.

### SUMMARY OF THE INVENTION

In one aspect, the present invention provides a crystal of a receptor-antibody complex comprising a receptor-antibody complex of an epidermal growth factor receptor (EGFR) extracellular domain and antibody 11F8 Fab, wherein the crystal has a resolution determined by X-ray crystallography of better than about 5.0 Angstroms. Preferably, the crystal has a resolution determined by X-ray crystallography of better than about 4.0 Angstroms, more preferably better than about 3.0 Angstroms.

In one embodiment, the crystal is of Fab11F8 and isolated domain 3 of EGFR, belongs to space group P2₁ and has unit cell dimensions a=154.4 Å, b=139.1 Å, c=175.3 Å, and β=90.02°. This crystal may have the atomic coordinates provided in Table 2.

In another embodiment, the crystal is of Fab11F8 and isolated domain 3 of EGFR (EGFRd3), belongs to space group C222₁ and has unit cell dimensions a=77.8 Å, b=70.9 Å, and c=147.1 Å. This crystal may have the atomic coordinates provided in Table 3.

In another aspect, the present invention provides a method for preparing a crystal of a complex of an epidermal growth factor receptor (EGFR) extracellular domain or isolated domain 3 thereof and antibody 11F8Fab comprising preparing a solution containing the extracellular domain of EGFR or domain 3 thereof and antibody 11F8 Fab fragment, and growing the crystal. Preferably the pH of the solution is about 6.0 to about 8.0.

In another aspect, the present invention provides a method of identifying a mimetic of antibody 11F8 comprising comparing a three-dimensional structure of the mimetic with a three-dimensional structure determined for one or both of the above-referenced crystal complexes. Thus, the three dimensional structure of the mimetic may be compared with at least a subset of the coordinates provided in Table 2 or Table 3.

In one embodiment, identifying a mimetic is carried out by comparing the three-dimensional structure of, the mimetic against the coordinates of at least one EGFR amino acid bound by antibody 11F8Fab. Such EGFR amino acid may be selected from the group consisting of Pro349, Gln384, His409, Ser418, Ile438, Ser440, Gly441, Lys443, Thr464, Lys465, Thr466, Ile467, Ser468, Asn469, Gly471, and Asn473. In one embodiment, the locations of atoms of the mimetic that contact EGFR correspond to atoms of antibody 11F8 that contact EGFR. In yet another embodiment, screening is carried out by comparing a three dimensional structure of a mimetic with the atomic coordinates of a region of EGFR selected from the group consisting of about amino acid residue 348 to about amino acid residue 354, about amino acid residue 380 to about amino acid residue 385, about amino acid residue 405 to about amino acid residue 420, about amino acid residue 435 to about amino acid residue 475 and combinations thereof.

The mimetic may be a small molecule, a peptide, or a polypeptide, such as an antibody or a functional fragment thereof.

In another aspect of the invention, a mimetic that is an antibody or a fragment thereof is identified by introducing one or more substitutions in at least a single CDR region of antibody 11F8 and/or at non-CDR amino acids of the antibody that interact with the CDR and affect its conformation. In one embodiment, at most a single substitution is made in each CDR. In another embodiment, substitution are made solely in CDR3 or at amino acids that affect the conformation of CDR3.

In another aspect, the present invention provides the above methods carried out with use of a computer.

The invention further provides a method for synthesizing the mimetic and assaying its binding or physiological activity to select EGFR antagonists useful for inhibiting EGFR function and treating EGFR-associated diseases or conditions. In an aspect of the invention, a mimetic is provided that inhibits tyrosine kinase activity of the receptor. In another aspect of the invention, the mimetic inhibits dimerization of EGFR expressed by a cell. Preferably, the mimetic blocks binding of EGF to EGFR. Mimetics of the invention bind to EGFR and inhibit EGFR functional activity, preferably to a similar or greater extent than antibody 11F8.

In another aspect, the present invention provides a computer-assisted method for identifying a mimetic of cetuximab comprising a processor, a data storage system, an input device, and an output device, comprising: inputting into the programmed computer through said input device data comprising the three-dimensional coordinates of at least a subset of the atoms of EGFR as set out in Table 2 or Table 3; providing a database of chemical and peptide structures stored in said computer data storage system; selecting from said database, using computer methods, structures having a portion that is structurally similar to said criteria data set; and outputting to said output device the selected chemical structures having a portion similar to said criteria data set.

In another aspect, the present invention provides a machine-readable medium having stored thereon a plurality of executable instructions to perform a method to identify a mimetic of cetuximab using a crystal of a receptor-antibody complex comprising a receptor-antibody complex of an epidermal growth factor receptor (EGFR) extracellular domain or isolated domain 3 thereof and antibody 11F8Fab, the method comprising: comparing a three-dimensional structure of a mimetic with a three dimensional structure an epidermal growth factor receptor (EGFR) extracellular domain (or domain 3 thereof) and antibody 11F8Fab having an X-ray crystallography resolution of better than about 5.0 Angstroms.

The EGFR coordinates may comprise at least a subset of the atomic coordinates of Table 2 or Table 3. In one embodiment, identifying a mimetic comprises comparing the three-dimensional structure of a mimetic with a three-dimensional structure of at least one EGFR amino acid bound by antibody 11F8Fab. In another embodiment identifying a mimetic comprises comparing a three dimensional structure of a mimetic with the atomic coordinates of a region of EGFR selected from the group consisting of about amino acid residue 348 to about amino acid residue 354, about amino acid residue 380 to about amino acid residue 385, about amino acid residue 405 to about amino acid residue 420, about amino acid residue 435 to about amino acid residue 475 and combinations thereof.

In another aspect, the present invention provides a machine-readable medium having stored thereon a plurality of executable instructions to perform a method for identifying a mimetic of antibody 11F8, the method comprising: introducing in silico substitutions in at least a single CDR region of antibody 11F8 to obtain a pool of variants; and using a computer and at least a subset of the EGFR coordinates provided in Table 2 or Table 3 to select a variant with desired EGFR binding characteristics.

In another aspect, the present invention provides a antibody 11F8 mimetic identified by any of the above methods.

In another aspect, the present invention provides a method of inhibiting EGFR comprising administering the identified mimetic.

In another aspect, the present invention provides a method of treating a disease or condition associated with EGFR expression comprising administering the identified mimetic. In one non-limiting embodiment, the present invention provides a method of inhibiting growth of a tumor cell that expresses EGFR comprising administering one or more above identified mimetics. In another embodiment, the present invention provides a method of treating a hyperproliferative diseases stimulated by a ligand of EGFR by administering one or more antibody 11F8 mimetics.

In another aspect, the present invention provides a method of treating psoriasis comprising administering one or more antibody 11F8 mimetics.

### BRIEF DESCRIPTION OF THE FIGURES

FIG. 1 illustrates the ligand-induced dimerization of EGFR.

FIGS. 2A and 2B illustrate Fab11F8 binding to sEGFR and inhibition of sEGFR binding to EGF thereby.

FIGS. 3A-D illustrate various aspects of Fab11F8 binding to domain III of sEGFR.

FIG. 4A-C illustrate features of the shared Fab11F8, FabC225 and EGF binding region on domain III.

FIGS. 5A-C show that Fab11F8 and FabC225 use distinct interactions to recognize a common surface on EGFR.

FIG. 6. shows an analysis of mutations in the sEGFRd3 binding site upon the affinity of sEGFR for Fab11F8, FabC225 and EGF.

FIGS. 7A-C show comparisons of the structures of Fab11F8 and FabC225.

FIGS. 8A-C compare the mechanisms of inhibition of EGFR activation by IMC-11F8 and cetuximab.

### DETAILED DESCRIPTION OF THE INVENTION

In an effort to generate fully human anti-EGFR antibodies that inhibit the receptor, a non-immunized human Fab phage display library containing 3.7×10¹⁰ unique clones (de Haard et al., 1999; Lu et al., 2004b) was screened for Fab fragments that would bind A431 epidermoid carcinoma cells (which express high levels of EGFR) (Lu et al., 2004b), and also compete with cetuximab for binding to the cell surface (Liu et al., 2004). Of four unique Fab clones that were selected, only one (termed 11F8) displayed a dose-dependent inhibitory effect on EGF stimulated EGFR activation in A431 cells (Liu et al., 2004). A fully human antibody bearing 11F8 antigen-combining regions (“IMC-11F8”) inhibits EGFR activation in several cell-lines (Liu et al., 2004; Lu et al., 2004b), blocks tumor growth in xenograft models (Lu et al., 2005; Prewett et al., 2004), and has performed well in phase I clinical trials (Kuenen et al., 2006). As a fully human antibody, antibody 11F8 has a significant advantage over the chimeric cetuximab antibody, which contains entirely mouse-derived sequences in its variable domains that are fused to human constant domains. Cetuximab (Erbitux®), which is approved for use in advanced colorectal cancer and head and neck squamous-cell carcinoma, elicits immune reactions (presumably against mouse antibody sequences) in ˜19% of cases (Lenz, 2007). As expected for a fully human antibody (Weiner, 2006), antibody 11F8 has shown no evidence of such immune hypersensitivity in clinical trials (Kuenen et al., 2006).

To establish the mechanism of EGFR inhibition by this fully-human therapeutic antibody, the X-ray crystal structures of the Fab fragment of antibody 11F8 bound to the full length EGFR extracellular region (sEGFR) and bound to isolated domain III of EGFR (EGFRd3) were determined. Despite being quite different in CDR sequences, 11F8 resembles cetuximab remarkably closely in the EGFR epitope that it recognizes, and therefore in its mode of EGFR inhibition. However, the details of the antibody/receptor interactions are quite different.

In one embodiment, the invention provides a co-crystal of Fab11F8 and isolated domain 3 of EGFR that belongs to space group P2₁ and that has unit cell dimensions of a=154.4 Å, b=139.1 Å, c=175.3 Å, and β=90.02°. This crystal may have the atomic coordinates provided in Table 2.

In another embodiment, invention provides a co-crystal of Fab11F8 and isolated domain 3 of EGFR (EGFRd3) that belongs to space group C222₁ and that has unit cell dimensions of a=77.8 Å, b=70.9 Å, and c=147.1 Å. This crystal may have the atomic coordinates provided in Table 3.

To obtain the crystal for which structural coordinates are shown Table 2, the entire extracellular region (i.e., amino acids 1-618 of mature EGFR, including domains I, II, III and IV) is used, plus a C-terminal hexa-histidine tag (Ferguson, K. M. et al., 2000, Embo J 19, 4632-4643; Ferguson, K. M. et al., 2003, Mol Cell 11, 507-517). (See GenBank Accession No. 1NQLA). 11F8 Fab contains the Fab fragment of antibody 11F8, i.e., the heavy and light chain variable region sequences of antibody 11F8 with IgG1 C_{H}1 heavy and kappa light chain constant domains. The CDR regions of the heavy chain of 11F8 have the following sequences: a CDR1 region with a sequence of SGDYYWS (SEQ ID NO:1), a CDR2 region with a sequence of YIYYSGSTDYNPSLKS (SEQ ID NO:2), and a CDR3 region with a sequence of VSIFGVGTFDY (SEQ ID NO:3). The CDR regions of the light chain of 11F8 have the following sequences: a CDR1 region with a sequence of RASQSVSSYLA (SEQ ID NO:4), a CDR2 region with a sequence of DASNRAT (SEQ ID NO:5), and a CDR3 region with a sequence of HQYGSTPLT (SEQ ID NO:6).

The sequences of the proteins in the crystal, i.e., 11F8 Fab and the extracellular domain of EGFR, are also reported with the atomic coordinates of Table 2.

Crystallization of the EGFR:antibody 11F8 Fab complexes may be carried out from a solution of antibody 11F8Fab and EGFR with various techniques, such as microbatch, hanging drop, sitting drop, sandwich drop, seeding and dialysis. The solution is prepared by combining EGFR extracellular domain with 11F8 Fab in a suitable buffer. A standard buffering agent such as Hepes, Tris, MES and acetate may be used. The buffer system may also be manipulated by addition of a salt such as sodium chloride, ammonium sulfate, sodium/potassium phosphate, ammonium acetate among others. Imidazole may also be used as a buffer. The concentration of the salt is preferably about 10 mM to about 500 mM, more preferably about 25 mM to about 100 mM, and most preferably about 50 mM. The pH of the buffer is preferably about 6 to about 8, more preferably about 7 to about 8. The concentration of the protein in the solution is preferably that of super-saturation to allow precipitation. The solution may optionally contain a protein stabilizing agent.

In one embodiment, the crystal is precipitated by contacting the solution with a reservoir that reduces the solubility of the proteins due to presence of precipitants, i.e., reagents that induce precipitation. Such contacting may be carried out through vapor diffusion. Examples of precipitants include ammonium sulfate, ethanol, 3-ethyl-2,4 pentanediol, and glycols, particularly polyethanol glycol (PEG). The PEG utilized preferably has a molecular weight of about 400 to about 20,000, more preferably about 3000 Da, with a concentration of about 10% to about 20%, more preferably about 15% (w/v). Some precipitants may act by making the buffer pH unfavorable for protein solubility.

The temperature during crystallization may be in the range of about 0° C. to about 30° C., such as about 20° C. to about 30° C., such as about 25° C. In addition to use in the determination of structure, the crystallization techniques of the invention may also be used to increase purity of proteins.

Precipitation may also be carried out in the presence of a heavy metal such as cadmium to further improve analysis of the crystal after precipitation. In one embodiment illustrated in the example, about 0.5 μl (or microliter) Fab11F8/sEGFR protein at 10 mg/ml in 25 mM Hepes, 50 mM NaCl, pH 7.5 is contacted with 0.5 μl (or microliter) reservoir solution of about 12% PEG 3350, about 1 M NaCl, about 50 mM MES and about pH 6.5. For Fab11F8/sEGFRd3 protein at 6 mg/ml in 25 mM Hepes, 50 mM NaCl, pH 7.5 is contacted with 0.5 μl (or microliter) reservoir solution of about 12% PEG 3350, about 250 mM ammonium sulfate, about 50 mM sodium acetate and about pH 5.

The atomic coordinates of the co-crystals of the present invention are disclosed in Table 2 (11F8 Fab: sEGFRd3) and Table 3 (11F8 Fab: sEFGR). Accordingly, the crystals and the deduced atomic coordinates allow for studying the binding interaction of antibody 11F8 with EGFR and EGFR inhibition and for comparison with the binding interactions of other EGFR-binding antibodies such as cetuximab. The three dimensional structures further allow for the identification of binding mimetics of antibody 11F8 by screening potential mimetics against at least part of the structure(s), such as against a subset of atoms provided in Table 2 or Table 3.

The three dimensional structures of the 11F8Fab:sEGFR and 11F8Fab:EGFRd3 complexes as defined by atomic coordinates are obtained from the X-ray diffraction pattern of each crystal and the electron density map derived therefrom. One method for determining the three dimensional structure is by molecular replacement which involves use of the structure of a closely related molecule or receptor ligand complex. An alternative method employs heavy atom derivatives.

One of skill in the art will also appreciate that the atomic coordinates provided are not precise, but are obtained from electron density measured for the crystal. Initial coordinates are determined by matching the protein backbone and side chains to the electron density map. The coordinates are refined by minimizing the overall energy of the protein (e.g., by adjusting bond lengths and angles), in view of the determined electron density. In some locations in the atomic structure, atoms of amino acid side chains may not be fully resolved due to, for example, solvent interactions and the like. Accordingly, the side chain that is modeled may differ from the actual side chain at that amino acid position. The present invention encompasses structures having root mean square deviations of backbone atoms of not more than about 1.5 Å, or more preferably not more than about 1.0 Å, or most preferably, not more than about 0.5 Å for residues of EGFR extracellular domain or 11F8 Fab that are used in identifying mimetics. The present invention encompasses variations within acceptable standards of error in the art for a crystal with the resolution disclosed herein.

It will also be appreciated that the origin of the atomic coordinates is arbitrarily defined. Accordingly, the same atomic structure can be represented by sets of coordinates that are numerically different, but that identify the same atomic positions. The present invention encompasses such alternative coordinate sets.

Various aspects of the invention are further described below with reference to the appended figures.

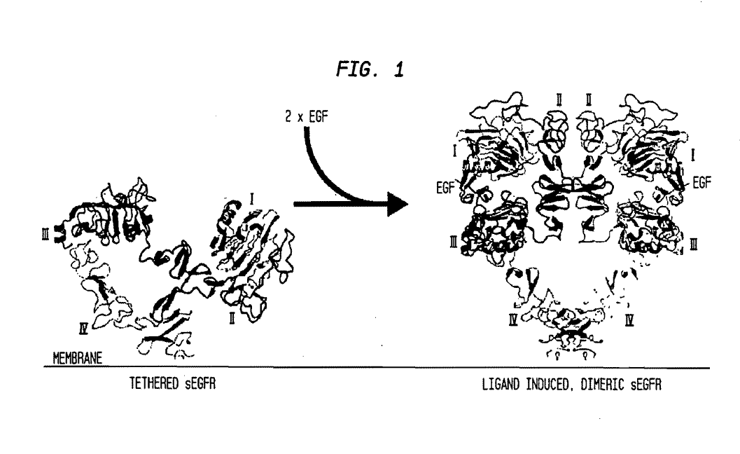

FIG. 1 illustrates the ligand-induced dimerization of EGFR. In the unliganded state EGFR exists as a tethered monomer (left hand cartoon view). Domain II (green) interacts with domain IV (white with elements of secondary structure highlighted in green); domains I (white with red highlights) and III (red) are far apart. The arrangement of the domains in the ligand induced, dimeric state (right hand cartoon view) is dramatically different. Domains I and III are much closer together and each interacts with the ligand (EGF, cyan). Local conformational changes in domain II stabilize the precise conformation of this domain required to form the entirely receptor mediated dimer. The colors of the right hand molecule in the dimer are lightened for contrast. Domain IV in dimer has been modeled on using the same domain III/IV relationship as seen in the tethered monomer. The grey line represents the approximate location of the membrane. This figure was generated using coordinates from pdb ids 1yy9, 1nql and 1ivo.

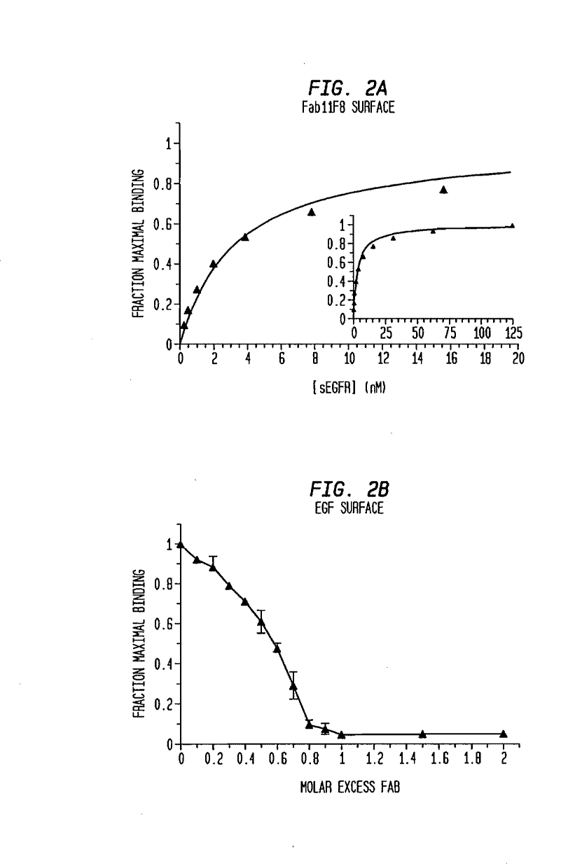

FIGS. 2A and 2B illustrate Fab11F8 binding to sEGFR and inhibition of sEGFR binding to EGF thereby.

FIG. 2A. illustrates a Surface Plasmon Resonance (SPR) analysis of sEGFR binding to immobilized Fab11F8. A series of sEGFR samples of the indicated concentrations were passed over a Biosensor surface to extra which Fab11F8 had been covalently coupled. A representative data set of the equilibrium SPR response for each sample, expressed as the fraction of the maximum binding, is plotted as a function of the concentration of sEGFR. The inset shows that no additional binding is seen at higher sEGFR concentrations. The curve indicates the fit to a simple one-site Langmuir binding equation for the data set shown. Mean KD value of 3.3±0.5 nM was obtained from at least three independent binding experiments.

FIG. 2B shows the ability of Fab11F8 to compete for sEGFR binding to immobilized EGF. The indicated molar excesses of Fab were added to samples of fixed concentration of sEGFR (600 nM) and these samples were passed over immobilized EGF. The equilibrium SPR responses obtained for each sample, expressed as a fraction of the response with no added Fab, is plotted as a function of the molar excess of Fab. All binding is abolished at a 1:1 stochiometry of Fab11F8/sEGFR and the IC50 value for these conditions is 100 nM.

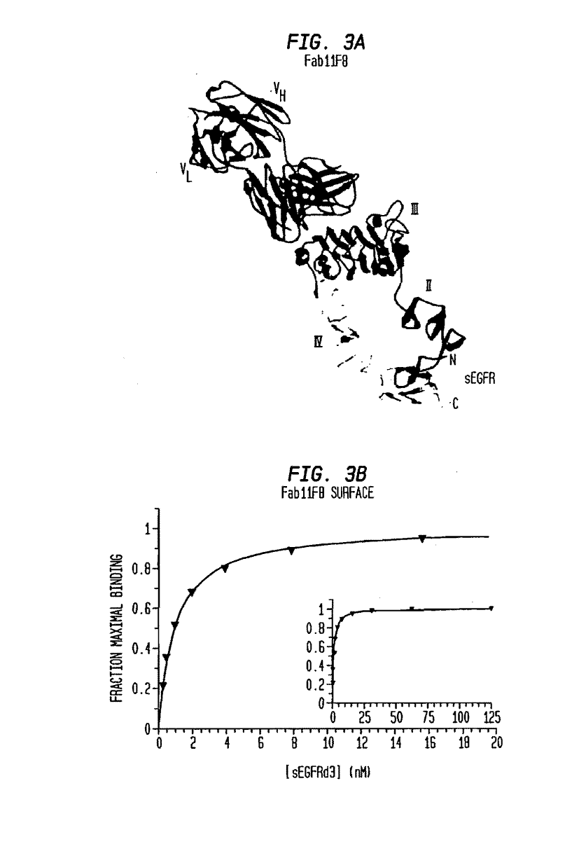

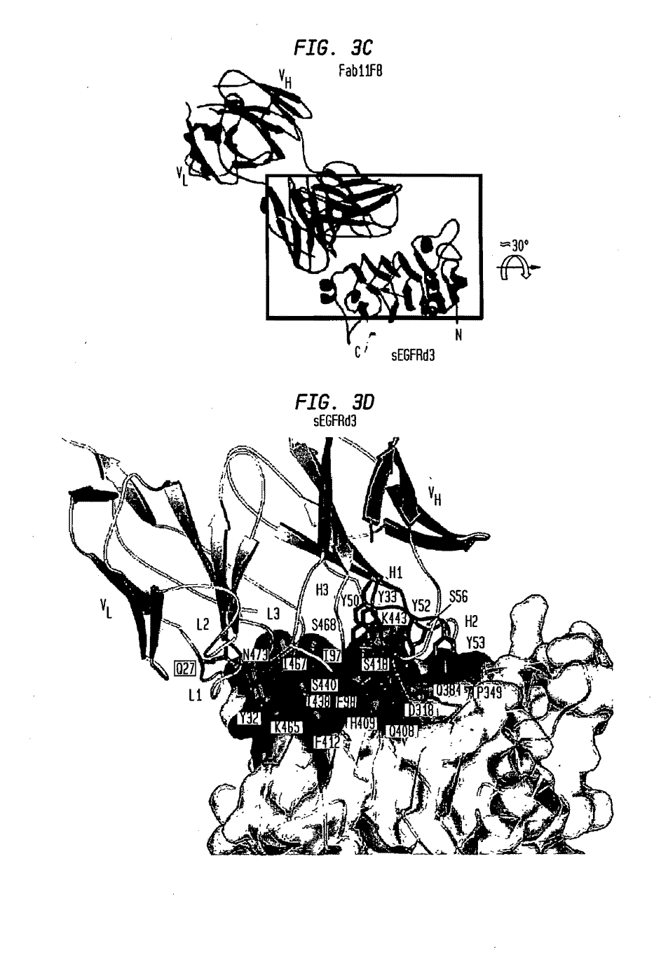

FIGS. 3A-D illustrate various aspects of Fab11F8 binding to domain III of sEGFR.

FIG. 3A is a cartoon representation of Fab11F8/sEGFR complex. The VL chain of Fab11F8 is shown in yellow, the VH chain in orange, domain III of sEGFR is in red, domain II in green and domain IV in grey with elements of secondary structure highlighted in green.

FIG. 3B shows a Surface Plasmon Resonance (SPR) analysis of sEGFRd3 binding to immobilized Fab11F8 performed and analyzed as described in FIG. 2A. A mean KD value of 1.0±0.1 nM was obtained.

FIG. 3C is a cartoon representation of Fab11F8/sEGFRd3 complex with domain III of sEGFR in the same orientation as in part A. Domains are colored as in part A.

FIG. 3D shows a detailed view of the interface between Fab11F8 and domain III of sEGFR. The view is an approximate 30° rotation about a horizontal axis with respect to part C. The VL and VH chains are shown with yellow and orange highlights on the elements of secondary structure. The parts of the CDRs that interact with domain III are colored yellow for CDRs L1 and L3, cyan for CDR H1 and orange for CDRs H2 and H3. Side chains that make direct hydrogen bond or key van der Waals contacts are shown in stick representation and are labeled and colored yellow for CDRs L1 and L3, cyan for CDR H1 and orange for CDR H2 and H3. The main chain of domain III of sEGFR is show in a grey cartoon highlighted in red. Side chains on domain III that make key contacts with the Fab are shown in green stick representation and labeled in black (see text for details). A transparent molecular surface is show around domain III. The darker grey shading indicates the region of this surface that is in contact (=1.4 Å) with Fab11F8. The interface atoms were defined using the program CNS (Brunger et al., 1998).

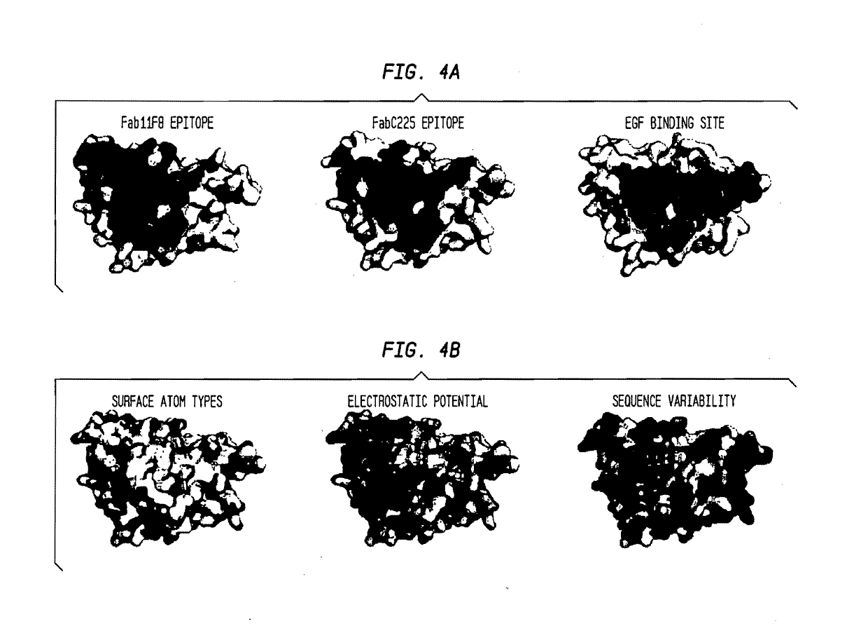

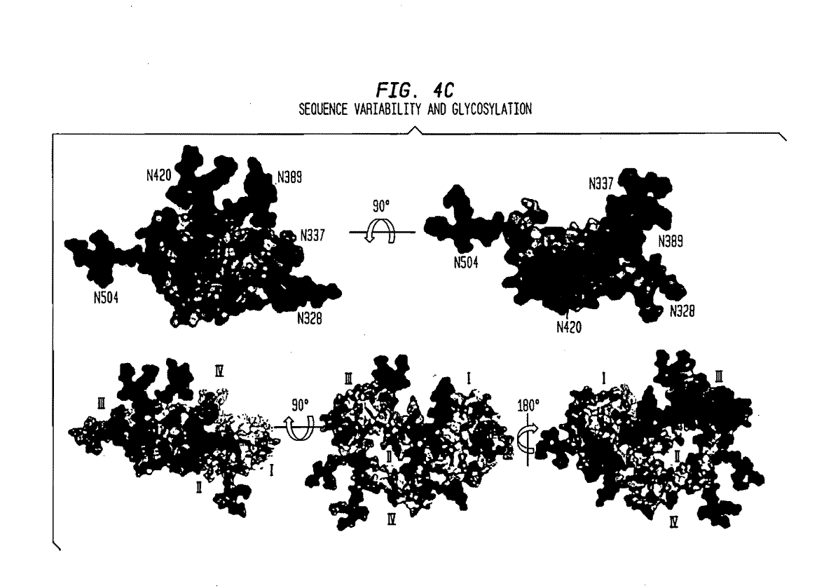

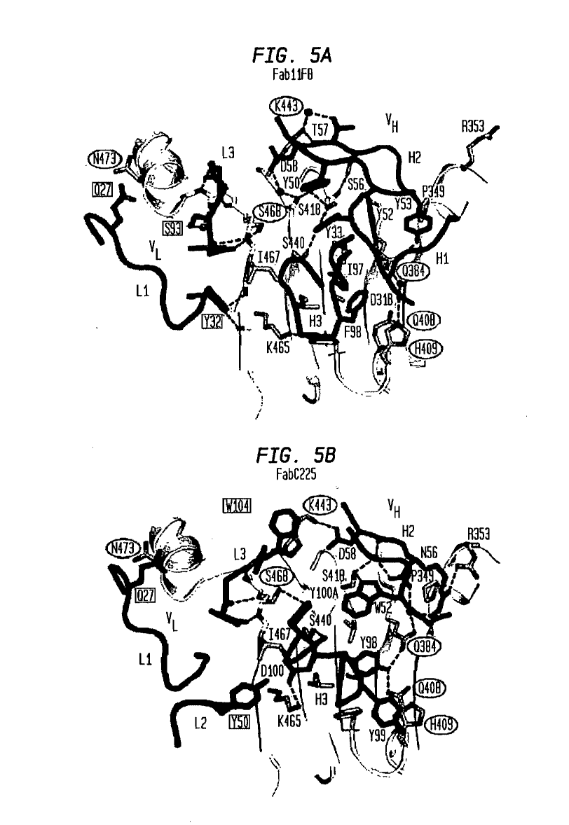

FIG. 4A-C illustrate features of the shared Fab11F8, FabC225 and EGF binding region on domain III.

FIG. 4A shows molecular surface representations of domain III of sEGFR. The colored areas indicate the contact regions (as defined in FIG. 3D) for Fab11F8 (red), FabC225 (yellow) and EGF (blue). Orientation is looking down onto the domain III binding site for these ligands, an approximate 90° rotation about the axis indicated in FIG. 3C.

FIG. 4B shows functional features mapped onto the domain III molecular surface. In the left panel the surface is colored by atom type; negative red, positive blue, polar oxygen pink or polar nitrogen light blue, and apolar white. In the middle panel the electrostatic potential ranging from −2.5 kT (red) to +2.5 kT is projected on to the same surface and in the right panel the degree of sequence variability is projected with dark green representing the least conserved surface exposed amino acids and white the most conserved. Electrostatic potential calculations were done using the Adaptive Poisson-Boltzmann Solver (APBS) implemented in Pymol (Baker et al., 2001; DeLano, 2004). Sequence variability was defined using the Consurf web served (Landau et al., 2005).

FIG. 4C shows a sequence variability profile for two orthogonal views of domain III (upper panel) and three orientations of sEGFR (lower panel). High mannose chains (yellow) have been placed at the each positions of glycosylation on sEGFR guided by the one or two ordered sugar groups that can been seen in the X-ray crystal structures.

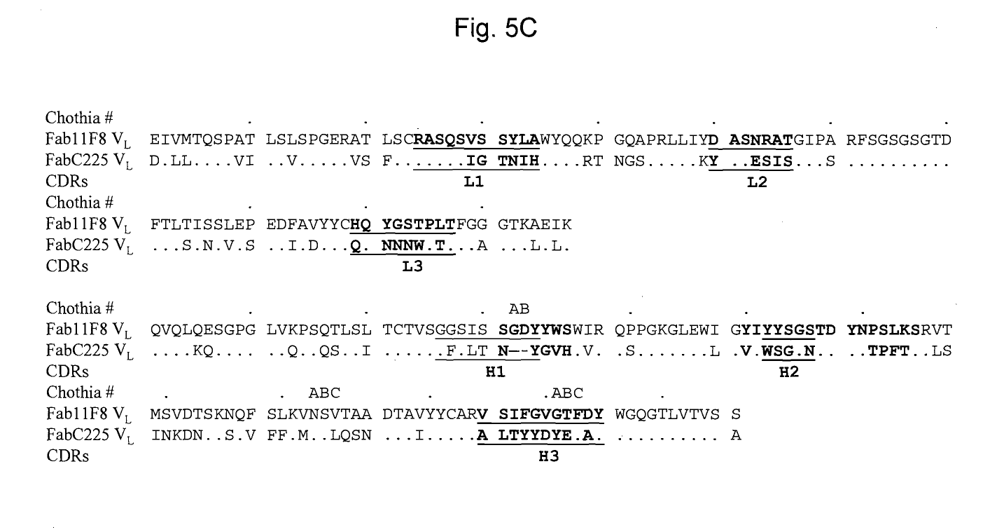

FIGS. 5A-C show that Fab11F8 and FabC225 use distinct interactions to recognize a common surface on EGFR.

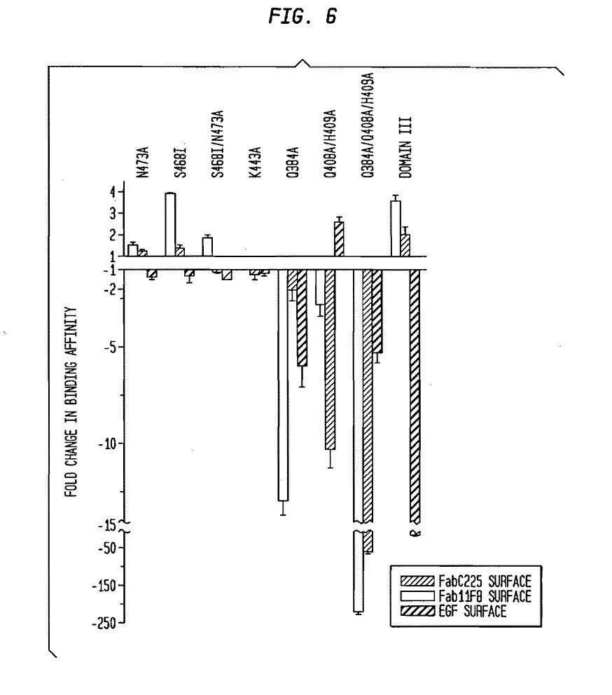

FIG. 5A shows a detailed view of the interactions of domain III of sEGFR with Fab11F8 and FIG. 5B shows the same for FabC225. The orientation is as in FIG. 4A. Only those parts of Fabs that are involved in binding are shown. The VL loops are colored in yellow, CDR H1 (Fab11F8 only) in cyan and CDRs H2 and H3 in orange. Side chains from the Fab that interact with sEGFR are shown in stick representation and labeled using this same color scheme. A cartoon for the binding site region of domain III of sEGFR is shown in white. Side chains from sEGFRd3 that interact with the Fab are show in stick representation in pink with black labels. The same set of side chains on the sEGFR are shown in both panels. For clarity the side chains that line the hydrophobic binding pocket on domain III (F412, A415, V417, and I438) are not labeled. Hydrogen bonds are shown with dotted black lines. Key water molecules are shown as green spheres. Side chains on sEGFRd3 that are altered in analysis of the binding site mutations (FIG. 6) are ringed. The Fab11F8/sEGFRd3 has 16 direct hydrogen bonds and 5 waters contributing to the interface while FabC225 interface has 10 direct hydrogen bonds and 2 well ordered water molecules.

FIG. 5C shows the amino acid sequence alignment of the variable domains of Fab11F8 and FabC225. Only non-identical amino acids are shown for FabC225 with a period indicating a position that is identical to Fab11F8 and a dash indicating a gap. Amino acids involved in interacting with domain III (side chain and/or main chain interactions) are highlighted with the same color scheme used in FIGS. 5A and 5B. The Chothia numbering scheme is indicated above the sequence with a dot above every 10th amino acid (Chothia and Lesk, 1987; Chothia et al., 1986). For these antibodies this numbering scheme is identical to the Kabat numbering scheme with the exception of the placement of the insertion in CDR H1. In this sequence alignment the two amino acid insertion in Fab11F8 is placed in the topologically more correct position after amino acid 31 of the heavy chain and given the numbers 31A and 31B (Chothia and Lesk, 1987; Chothia et al., 1986). Kabat numbering would place the insertion after amino acid 35. Kabat defined CDR's are indicated in bold and Chothia defined CDR's are underlined. The Chothia numbering scheme is used in all text and figures. It should be noted that in the submitted pdb files and in Li et al (Li et al., 2005) the amino acids are numbered sequentially for each Fab.

FIG. 6. shows an analysis of mutations in the sEGFRd3 binding site upon the affinity of sEGFR for Fab11F8, FabC225 and EGF. The fold change in binding affinity for each indicated altered form of sEGFR to immobilized Fab11F8 (yellow), FabC225 (magenta) and EGF (black) is shown. KD values for binding of wild type sEGFR, determined using SPR analysis as described in FIG. 2, were 3.3±0.5 nM (Fab11F8), 2.3±0.5 nM (FabC225) and 130±4 nM (EGF). Fold change is shown relative to these numbers with positive (upward) fold change for those that bind more tightly and a negative (downward) fold changes for those that bind more weakly. Errors indicate the standard deviation for at least three separate measurements.

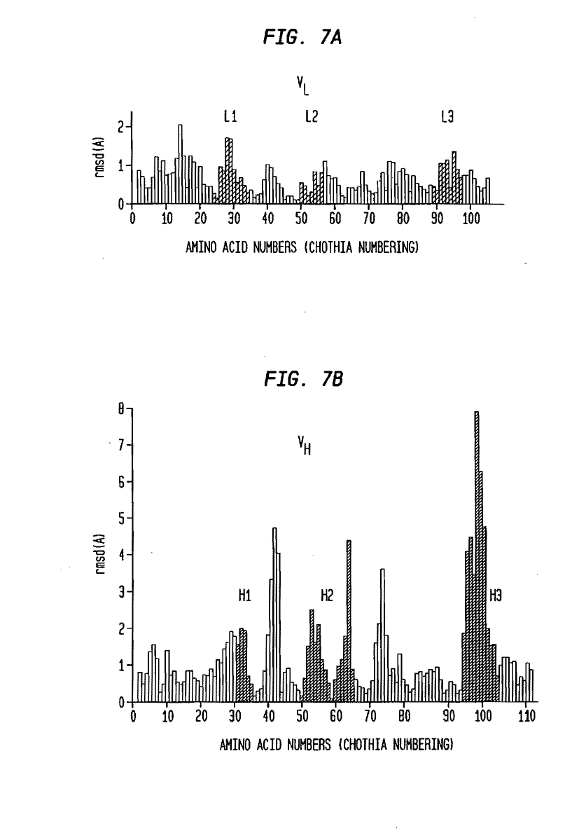

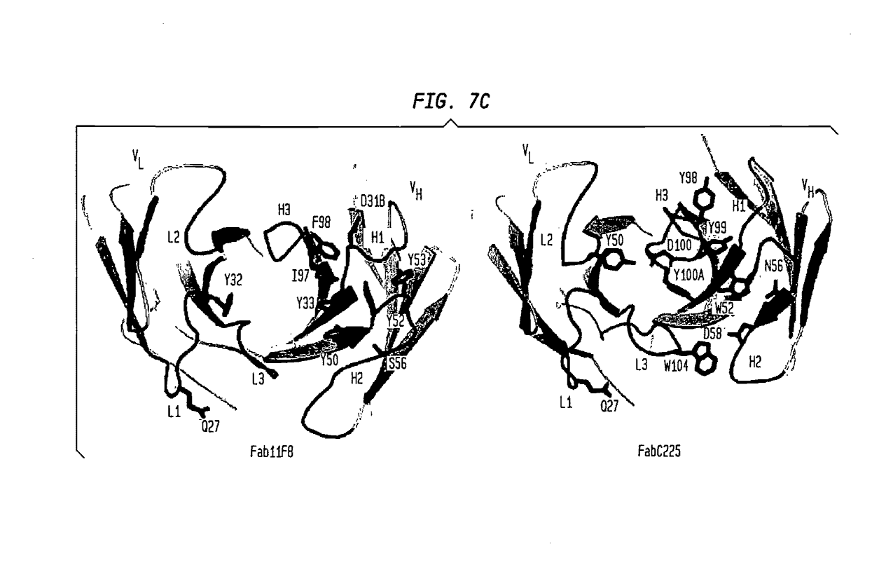

FIGS. 7A-C show comparisons of the structures of Fab11F8 and FabC225. The root mean square deviation (rmsd) of C□ positions between Fab11F8 and FabC225 for the variable portion of the light chains are shown in FIG. 7A, and for the variable portion of the heavy chains in FIG. 7B. All main chain atoms for each pair of chains were individually superimposed using the program Superpose (CCP4, 1994). The CDRs are marked and highlighted in yellow for the VL and orange for the VH.

FIG. 7C is a cartoon representation of the variable domains of Fab11F8 and FabC225 looking up from the domain III binding site and with a common orientation with respect to domain III in each complex. The entire CDR loops (Kabat definition) are highlighted in dark grey. Key side chains that interact with domain III are shown in stick representation and colored yellow for the VL CDRs, cyan for CDR H1 and orange for CDRs H2 and H3. The similarity in the position of each VL chain over domain III can be seen, as can the similarity in the VL CDR conformations. The VH chain of FabC225 is rotated slightly counterclockwise relative to the VL chain compared to the position of the VH chain of Fab11F8. The differences in CDR conformation and amino acid composition of the two Fabs can be appreciated in this view.

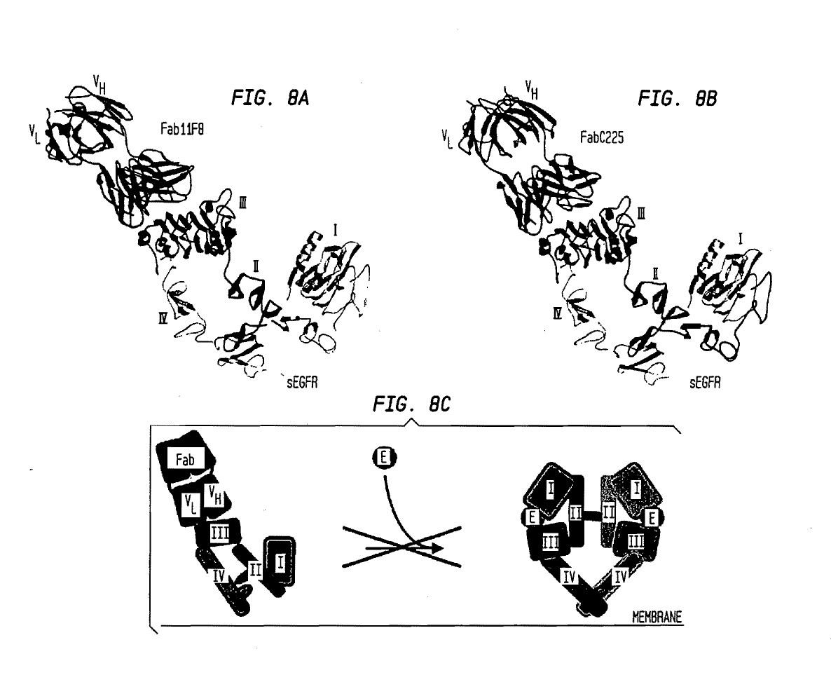

FIGS. 8A-C compare the mechanisms of inhibition of EGFR activation by antibody 11F8 and cetuximab.

FIG. 8A is a cartoon model of Fab11F8 bound to sEGFR colored as in FIG. 3A. Domain I and the N-terminal portion of domain II (grey) have been modeled onto the structure of Fab11F8/sEGFR using the coordinates from the FabC225/sEGFR complex.

FIG. 8B is a cartoon representation of FabC225/sEGFR complex (pdb id 1yy9) colored as in FIG. 8A.

FIG. 8C illustrates a model for the mechanism of inhibition of ligand induced dimerization and activation of EGFR for IMC-11F8 and cetuximab based on the structures presented here and in Li et al (Li et al., 2005). The binding of the antibody to domain III of EGFR prevents ligand binding and may also sterically inhibit the conformational change that must occur for dimerization.

Identification of mimetics of antibody 11F8 may be carried out with only a subset of the coordinates provided, such as those of amino acid residues of EGFR or antibody 11F8Fab that are associated in the complex.

Potential mimetics are examined against EGFR, particularly one or more of the above residues, through the use of computer modeling using a docking program. Such computer modeling allows for obtaining a positive initial indication of binding before synthesis and testing of the compound. If the testing shows sufficient interaction, then the compound may be synthesized and tested as a potential candidate. There is no limitation to the source of potential mimetics. For example, potential mimetics include structural databases of small molecules and other ligands represented in silico, as well as commercially available libraries of small molecules that can be similarly modeled. Potential mimetics further include peptides and macromolecules such as proteins, polypeptides, preferably antibodies or antibody fragments, synthetic polymer backbones having amino acid-like functional groups, and the like. Such potential mimetics may have defined structure, or be modeled on the basis of their similarity to other macromolecules of known structure. Iterative methods may be employed to vary one or more of the functional groups to improve the fit of the potential mimetic with EGFR. Those substances identified as mimetics, if not otherwise available to be tested for EGFR antagonist activity, may be synthesized.

In preferred embodiments, the locations of at least some atoms of antibody 11F8 mimetics that contact EGFR correspond to the locations of atoms of cetuximab that contact EGFR. The correspondence is preferably within about 2.0 Å, more preferably within about 1.0 Å, and most preferably with about 0.5 Å. The atoms usually interact with EGFR in a manner similar to the corresponding atoms of antibody 11F8Fab (i.e., polar, basic, acidic, hydrophobic). The mimetics may contain various numbers of such corresponding atoms, and binding of the mimetic to EGFR may be completely or only partially dependent on such corresponding interactions. In certain embodiments, such atomic interactions with EGFR may be supplemented by interactions of other atoms of the mimetic that also interact with EGFR. The binding ability of the mimetics can be evaluated by various computer programs as disclosed herein.

Docking may be accomplished by using software such as Quanta and Sybyl (manual model building software), followed by energy minimization and molecular dynamics with standard molecular mechanics force fields, such as CHARMM and AMBER. Specialized programs for docking include GRAM, GRID, Flexx, Glide, GOLD, MCSS, DOCK or AUTODOCK (See e.g. U.S. Pat. Nos. 5,856,116 and 6,087,478; Jorgensen W. L., 2004, Science 303, 1813-1818). Such procedure includes computer fitting of potential antagonists to EGFR to determine how the three dimensional structure of EGFR and the chemical properties of each amino acid interfere with EGFR activation, and to estimate attraction, repulsion and steric hindrance of the binding. Generally, tighter fits are preferred in that they are more likely to be effective when administered in vivo, and would be more selective for EGFR, minimizing binding to other receptors. Many of these programs also consider adsorption, distribution, metabolic and excretion characteristics of the molecules.

The docking program may be connected to a structure generator (such as SYNOPSIS) to perform de novo screening. An alternative to de novo screening, is creation of structures based on the binding site such as with programs including LUDI, SPROUT and BOMB, which allow a user to put a substituent in a binding site and then build up the substituent (Jorgensen W. L., 2004).

One of skill in the art would appreciate that the above screening methods may also be carried out manually, by building an actual three dimensional model based on the coordinates, and then determining desirable antagonists based on that model visually.

Of particular interest for designing mimetics are those amino acids that overlap with the binding site of EGR or TGF-α to EGFR. Such binding may interfere with the ligand-induced dimerization of the receptor or inhibit binding of the ligand to EGFR altogether.

Domains I and III of EGFR are responsible for binding of EGF to the receptor, and are of interest in designing antagonists. Of the amino acids of EGFR, some are involved in direct hydrogen bonding with 11F8 Fab. These amino acids include Gln384, H409, Ser418, Ser440, Gly441, Lys443, Thr464, Ile466, Ser468, and/or Asn469. Thr464 and Ile466 are involved in main-chain hydrogen bonds, i.e., the nature of the side chain is not directly relevant. Antagonists may be designed to bind to a few, most or none of these amino acids. Other amino acids of EGFR are in contact to some lesser degree with 11F8 Fab. These amino acids include: Leu348, Pro349, Arg353, Gln408, Phe412, Val417, Ile438, Lys465, Ile467, Gly471, and Asn473. Of the nine amino acids between 465 and 473, seven of them are in some contact with 11F8 Fab, this region of EGFR is ideal for screening of antagonists.

11F8 Fab does not bind to amino acids at positions 325, 346, 350, 354-357 and 411, despite these amino acids being involved in EGF/TGF-α binding. Screening may be carried out against these positions, or only for the positions bound by 11F8 Fab, or both. If screening is carried out based on the binding of 11F8 Fab to EGFR, such screening may be carried out in regions of amino acids of about 350 to about 354, amino acids of about 380 to about 385, amino acids of about 405 to about 420, amino acids of about 435 to about 475 and combinations thereof. One of skill in the art would appreciate that screening may simply be carried out against domains I and III of EGFR based on the crystal structure provided, and general area of the binding pocket, without focus on any particular amino acids bound by 11F8 Fab and/or ligands.

The mimetics, both peptides and small organic molecules, such as antibody and antibody fragments, bind to EGFR and mimic effects of antibody 11F8 both in vivo and in vitro. In addition to peptides and small organic molecules, the mimetic may be a sugar. The mimetic may also be a combination of peptides/small molecules/sugars, such as a peptide having a synthetic backbone. The mimetic may be designed based on criteria such as affinity for EGFR, desirable efficacy and/or desirable selectivity. These mimetics have at least a single physiological or binding activity of 11F8, which activity can be tested by assays provided further below.

As used herein, “mimetics” include antibody 11F8 mimetics with modifications that retain specificity for EGFR. Such modifications include, but are not limited to, conjugation to an effector molecule such as a chemotherapeutic agent (e.g., cisplatin, taxol, doxorubicin) or cytotoxin (e.g., a cytotoxic protein, or a non-protein organic chemotherapeutic agent). The mimetics can be modified by conjugation to detectable reporter moieties. Also included are mimetics with alterations that affect non-binding characteristics such as half-life (e.g., PEGylation).

Proteins and non-protein agents may be conjugated to the mimetics, such as by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990) for the conjugation of doxorubicin and those described by Amon et al., Adv. Exp. Med. Biol. 303, 79-90 (1991) and by Kiseleva et al., Mol. Biol. (USSR)25, 508-514 (1991) for the conjugation of platinum compounds.

In one embodiment, a library of small organic molecules is used to screen for mimetics in silico. In another embodiment, antibody 11F8 is used as a starting candidate, and varied to generate an antibody 11F8 variant with desirable properties. Such variant of antibody 11F8 may be a scFv, a Fab, diabody, or IgG. For example, conservative amino acid substitutions may be made at one or more of residues of antibody 11F8Fab which bind EGFR: light chain (LC) residues Tyr32, Tyr91, Thr94; heavy chain (HC) residues Asp33, Tyr35, Tyr52, Tyr54, Tyr55, His58, Thr59, Ile102.

A conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical conservative amino acid substitutions may be made within each of the following groups of amino acids:

(a.) glycine (G), alanine (A), valine (V), leucine (L) and isoleucine (I);

(b.) aspartic acid (D) and glutamic acid (E);

(c.) alanine (A), serine (S) and threonine (T);

(d.) histidine (H), lysine (K) and arginine (R):

(e.) asparagine (N) and glutamine (Q);

(f.) phenylalanine (F), tyrosine (Y) and tryptophan (W).

If the binding is not as tight in regard to one or more of the residues, less conservative substitutions may be made at those residues to optimize the binding. For example, an amino acid with a hydrophilic group may be substituted for one with a hydrophobic group.

In one embodiment, a mixture of all or some amino acids is introduced to synthesize variants of 11F8 randomly at specified positions in silico: Tyr32 (LC), Asp33 (HC), Tyr52 (HC), Tyr54 (HC), Tyr55 (HC), Ser58 (HC), and Thr59 (HC) of 11F8. These amino acid residues are involved in side chain hydrogen bonds, and thus are candidates for specific mutations aimed at modifying direct interactions. Such variation, where all 20 amino acids are used, would result in about 20⁷ variants which can then be screened. If only conservative substitutions are made, the variation would be much less, about 3⁷. Conservative and non-conservative substitutions at other positions in the CDRs of 11F8 that do not bind to EGFR directly should also be considered. For example, direct interactions between contact residues (e.g., main chain-main chain, main chain-side chain, side chain-side chain contacts) can be modified by introducing changes at amino acid positions that affect the position of 11F8 side chain and main chain atoms involved in direct interactions with EGFR. In one embodiment, at most a single substitution is made in each CDR. In another embodiment, a single substitution is made in the heavy chain CDR3 region of 11F8.

After such screening and selection, the selected mimetic may be synthesized, and various assays carried out to measure the biological or physiological activity of the mimetic to select an EGFR antagonist. A preferred EGFR antagonist has one or more of the following properties: inhibits EGFR tyrosine kinase activity; blocks ligand binding to EGFR; inhibits EGFR dimerization (homodimerization with EGFR or heterodimerization with another EGFR family receptor subunit); inhibits EGFR substrate phosphorylation; inhibits EGFR mediated gene activation; inhibits growth or proliferation of a cell the expresses EGFR. Preferably, the antagonist has substantially similar or improved effectiveness as an EGFR antagonist as compared to antibody 11F8.

Tyrosine kinase inhibition can be determined using well-known methods; for example, by measuring the autophosphorylation level of recombinant kinase receptor, and/or phosphorylation of natural or synthetic substrates. Thus, phosphorylation assays are useful in determining EGFR antagonists of the present invention. Phosphorylation can be detected, for example, using an antibody specific for phosphotyrosine in an ELISA assay or on a western blot. Some assays for tyrosine kinase activity are described in Panek et al., J. Pharmacol. Exp. Thera. (1997) 283: 1433-44 and Batley et al., Life Sci. (1998) 62: 143-50.

In addition, methods for detection of protein expression can be utilized to determine EGFR antagonists, wherein the proteins being measured are regulated by EGFR tyrosine kinase activity. These methods include immunohistochemistry (IHC) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA. See, e.g., Grandis et al., Cancer, (1996) 78:1284-92; Shimizu et al., Japan J. Cancer Res., (1994) 85:567-71; Sauter et al., Am. J. Path., (1996) 148:1047-53; Collins, Glia, (1995) 15:289-96; Radinsky et al., Clin. Cancer Res., (1995) 1:19-31; Petrides et al., Cancer Res., (1990) 50:3934-39; Hoffmann et al., Anticancer Res., (1997) 17:4419-26; Wikstrand et al., Cancer Res., (1995) 55:3140-48.

The ability of a mimetic to block ligand binding can be measured, for example, by an in vitro competitive binding assay, such as those known in the art. In this type of assay, a ligand of EGFR such as EGF is immobilized, and a binding assay is carried to determine the effectiveness of the mimetic to competitively inhibit binding of EGFR to the immobilized ligand.

In vivo assays can also be utilized to determine EGFR antagonists. For example, receptor tyrosine kinase inhibition can be observed by mitogenic assays using cell lines stimulated with receptor ligand in the presence and absence of inhibitor. For example, A431 cells (American Type Culture Collection (ATCC), Rockville, Md.) stimulated with EGF can be used to assay EGFR inhibition. Another method involves testing for inhibition of growth of EGFR-expressing tumor cells, using for example, human tumor cells injected into a mouse. See U.S. Pat. No. 6,365,157 (Rockwell et al.).

The present invention provides for coordinates of the co-crystal of the present invention on a computer readable format such as a magnetic disk, CD-ROM or a hard drive.

In another aspect, the present invention provides methods of treating EGFR-dependent diseases and conditions in mammals by administering a therapeutically effective amount of a mimetic of 11F8. One skilled in the art would easily be able to diagnose such conditions and disorders using known, conventional tests. Treatment means any treatment of a disease in an animal and includes: (1) preventing the disease from occurring in a mammal which may be predisposed to the disease but does not yet experience or display symptoms of the disease; e.g., prevention of the outbreak of the clinical symptoms; (2) inhibiting the disease, e.g., arresting its development; or (3) relieving the disease, e.g., causing regression of the symptoms of the disease. Therapeutically effective amount for the treatment of a disease means that amount which, when administered to a mammal in need thereof, is sufficient to effect treatment, as defined above, for that disease. A antibody 11F8 mimetic of the invention may be administered with an antineoplastic agent such as, for example, a chemotherapeutic.

Antibody 11F8 mimetics of the present invention are useful for treating tumors that express EGFR. EGFR expressing tumors are characteristically sensitive to EGF present in their environment, and can further be stimulated by tumor produced EGF or TGF-α. While not intending to be bound to any particular mechanism, the diseases and conditions that may be treated or prevented by the present methods include, for example, those in which tumor growth is stimulated through an EGFR paracrine and/or autocrine loop. The method is therefore effective for treating a solid tumor that is not vascularized, or is not yet substantially vascularized.

In another aspect of the invention, antibody 11F8 mimetics are used to inhibit tumor-associated angiogenesis. EGFR stimulation of vascular endothelium is associated with vascularization of tumors. Typically, vascular endothelium is stimulated in a paracrine fashion by EGF and/or TGF-α from other sources (e.g., tumor cells). Accordingly, the antibody 11F8 mimetics are effective for treating subjects with vascularized tumors or neoplasms.

Tumors that may be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued. The tumors may express EGFR at normal levels or they may overexpress EGFR at levels, for example, that are at least 10, 100, or 1000 times normal levels.

Examples of tumor that express EGFR and are stimulated by a ligand of EGFR include carcinomas, gliomas, sarcomas, adenocarcinomas, adenosarcomas, and adenomas. Such tumors can occur in virtually all parts of the body, including, for example, breast, heart, lung, small intestine, colon, spleen, kidney, bladder, head and neck, ovary, prostate, brain, pancreas, skin, bone, bone marrow, blood, thymus, uterus, testicles, cervix or liver. Some tumors observed to overexpress EGFR that may be treated according to the present invention include, but are not limited to, colorectal and head and neck tumors, especially squamous cell carcinoma of the head and neck, brain tumors such as glioblastomas, and tumors of the lung, breast, pancreas, esophagus, bladder, kidney, ovary, cervix, and prostate. Non-limiting examples of tumors observed to have constitutively active (i.e., unregulated) receptor tyrosine kinase activity include gliomas, non-small-cell lung carcinomas, ovarian carcinomas and prostate carcinomas. Other examples of tumors include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma.

The present invention also provides a method of treating a non-cancer hyperproliferative disease in a mammal comprising administering to the mammal an effective amount of the antibody of the present invention. As disclosed herein, “hyperproliferative disease” is defined as a condition caused by excessive growth of non-cancer cells that express a member of the EGFR family of receptors. The excess cells generated by a hyperproliferative disease express EGFR at normal levels or they may overexpress EGFR.

The types of hyperproliferative diseases that can be treated in accordance with the invention are any hyperproliferative diseases that are stimulated by a ligand of EGFR or mutants of such ligands. Examples of hyperproliferative disease include psoriasis, actinic keratoses, and seborrheic keratoses, warts, keloid scars, and eczema. Also included are hyperproliferative diseases caused by virus infections, such as papilloma virus infection. For example, psoriasis comes in many different variations and degrees of severity. Different types of psoriasis display characteristics such as pus-like blisters (pustular psoriasis), severe sloughing of the skin (erythrodermic psoriasis), drop-like dots (guttae psoriasis) and smooth inflamed lesions (inverse psoriasis). The treatment of all types of psoriasis (e.g., psoriasis vulgaris, psoriasis pustulosa, psoriasis erythrodermica, psoriasis arthropathica, parapsoriasis, palmoplantar pustulosis) is contemplated by the invention.

Administering the antibody 11F8 mimetic includes delivering the mimetic to a mammal by any method that may achieve the result sought. The term mammal as used herein is intended to include, but is not limited to, humans and mammalian laboratory animals, domestic pets and farm animals. The mimetic may be administered, for example, orally, parenterally (intravenously or intramuscularly), topically, transdermally or by inhalation. Topical administration may be preferred for certain hyperproliferative disorders.

In an embodiment of the invention, cetuximab mimetic can be administered in combination with one or more other anti-neoplastic agents, such as chemotherapeutic agents. Radiation can also be employed. For examples of combination therapies, see, e.g., U.S. Pat. No. 6,217,866 (Schlessinger et al.) (Anti-EGFR antibodies in combination with anti-neoplastic agents); WO 99/60023 (Waksal et al.) (Anti-EGFR antibodies in combination with radiation). Any suitable anti-neoplastic agent can be used, such as a chemotherapeutic agent, radiation or combinations thereof. The anti-neoplastic agent can be an alkylating agent or an anti-metabolite. Examples of alkylating agents include, but are not limited to, cisplatin, cyclophosphamide, melphalan, and dacarbazine. Examples of anti-metabolites include, but not limited to, doxorubicin, daunorubicin, paclitaxel, irinotecan (CPT-11), and topotecan. When the agent is radiation, the source of the radiation can be either external (external beam radiation therapy—EBRT) or internal (brachytherapy—BT) to the patient being treated. The dosage administered depends on numerous factors, including, for example, the type of agent, the type and severity tumor being treated and the route of administration of the agent. It should be emphasized, however, that the present invention is not limited to any particular dose.

For treatment of hyperproliferative disease, the antibody 11F8 mimetic can be combined with any conventional treatment agent. For example, when the hyperproliferative disease is psoriasis, there are a variety of conventional systemic and topical agents available. Systemic agents for psoriasis include methotrexate, and oral retinoids, such as acitretin, etretinate, and isotretinoin. Other systemic treatments of psoriasis include hydroxyurea, NSAIDS, sulfasalazine, and 6-thioguanine. Antibiotics and antimicrobials can be used to treat or prevent infection that can cause psoriasis to flare and worsen. Topical agents for psoriasis include anthralin, calcipotriene, coal tar, corticosteroids, retinoids, keratolytics, and tazarotene. Topical steroids are one of the most common therapies prescribed for mild to moderate psoriasis. Topical steroids are applied to the surface of the skin, but some are injected into the psoriasis lesions.

Hyperproliferative disease treatments further include administration of the cetuximab mimetic in combination with phototherapy. Phototherapy includes administration of any wavelength of light that reduces symptoms of the hyperproliferative disease, as well as photoactivation of a chemotherapeutic agent (photochemotherapy). For further discussion of treatment of hyperproliferative disorders, see WO 02/11677 (Teufel et al.) (Treatment of hyperproliferative diseases with epidermal growth factor receptor antagonists).

In certain embodiments of the invention, cetuximab mimetics of the invention can be administered with EGFR antagonists and/or antagonists of other receptors involved in tumor growth or angiogenesis. The receptor antagonists may bind to the receptor or the ligand to block receptor-ligand binding, or the receptor antagonists may otherwise neutralize the receptor tyrosine kinase. Ligands of EGFR include, for example, EGF, TGF-α amphiregulin, heparin-binding EGF (HB-EGF) and betacellulin. EGF and TGF-α are thought to be the main endogenous ligands that result in EGFR-mediated stimulation, although TGF-α has been shown to be more potent in promoting angiogenesis. Accordingly, EGFR antagonists include antibodies that bind to such ligands and thereby block binding to and activation of EGFR.

The antibody 11F8 mimetic may be used in combination with a VEGFR antagonist. In one embodiment of the invention, a antibody 11F8 mimetic is used in combination with a receptor antagonist that binds specifically to VEGFR-2/KDR receptor (PCT/US92/01300, filed Feb. 20, 1992; Terman et al., Oncogene 6: 1677-1683 (1991)). In another embodiment of the invention, a antibody 11F8 mimetic is used in combination with a receptor antagonist that binds specifically to VEGFR-1/Flt-1 receptor (Shibuya M. et al., Oncogene 5, 519-524 (1990)). In another embodiment, a antibody 11F8 mimetic is used in combination with a receptor antagonist that binds to a VEGFR ligand. For example, Avastin® (bevacizumab) is an antibody that binds VEGF. Particularly preferred are antigen-binding proteins that bind to the extracellular domain of VEGFR-1 or VEGFR-2 and block binding by ligand (VEGF or P1GF), and/or neutralize VEGF-induced or P1GF-induced activation. For example, Mab IMC-1121 binds to soluble and cell surface-expressed KDR. Mab IMC-1121 comprises the V_{H} and V_{L} domains obtained from a human Fab phage display library. (See WO 03/075840) In another example, ScFv 6.12 binds to soluble and cell surface-expressed Flt-1. ScFv 6.12 comprises the V_{H} and V_{L} domains of mouse monoclonal antibody MAb 6.12. A hybridoma cell line producing MAb 6.12 has been deposited as ATCC number PTA-3344.

In another embodiment, a antibody 11F8 mimetic is administered with an antagonist of insulin-like growth factor receptor (IGFR). In certain tumor cells, inhibition of EGFR function can be compensated by upregulation of other growth factor receptor signaling pathways, and particularly by IGFR stimulation. Further, inhibition of IGFR signaling results in increased sensitivity of tumor cells to certain therapeutic agents. Stimulation of either EGFR or IGFR results in phosphorylation of common downstream signal transduction molecules, including Akt and p44/42, although to different extents. Accordingly, in an embodiment of the invention, an IGFR antagonist (e.g., an antibody that binds to IGF or IGFR and neutralizes the receptor) is coadministered with a antibody 11F8 mimetic of the invention, thereby blocking a second input into the common downstream signaling pathway (e.g., inhibiting activation of Akt and/or p44/42). An example of a human antibody specific for IGFR is IMC-A12 (See WO 2005/016970).

Other examples of growth factor receptors involved in tumorigenesis against which antagonists may be directed are the receptors for platelet-derived growth factor (PDGFR), hepatocyte growth factor (HGFR), nerve growth factor (NGFR), fibroblast growth factor (FGFR), and macrophage stimulating protein (RON).

The antibody 11F8 mimetics can also be administered with intracellular RTK antagonists that inhibit activity of RTKs or their associated downstream signaling elements that are involved in tumor growth or tumor-associated angiogenesis. The intracellular RTK antagonists are preferably small molecules. Some examples of small molecules include organic compounds, organometallic compounds, salts of organic compounds and organometallic compounds, and inorganic compounds. Atoms in a small molecule are linked together via covalent and ionic bonds; the former is typical for small organic compounds such as small molecule tyrosine kinase inhibitors and the latter is typical of small inorganic compounds. The arrangement of atoms in a small organic molecule may represent a chain, e.g. a carbon-carbon chain or carbon-heteroatom chain or may represent a ring containing carbon atoms, e.g. benzene or a polycyclic system, or a combination of carbon and heteroatoms, i.e., heterocycles such as a pyrimidine or quinazoline. Although small molecules can have any molecular weight, they generally include molecules that would otherwise be considered biological molecules, except their molecular weight is not greater than 650 D. Small molecules include both compounds found in nature, such as hormones, neurotransmitters, nucleotides, amino acids, sugars, lipids, and their derivatives as well as compounds made synthetically, either by traditional organic synthesis, bio-mediated synthesis, or a combination thereof. See e.g. Ganesan, Drug Doscov. Today 7(1): 47-55 (January 2002); Lou, Drug Discov. Today, 6(24): 1288-1294 (December 2001).

More preferably, the small molecule to be used as an intracellular RTK antagonist according to the present invention is an intracellular EGFR antagonist that competes with ATP for binding to EGFR's intracellular binding region having a kinase domain or to proteins involved in the signal transduction pathways of EGFR activation. Examples of such signal transduction pathways include the ras-mitogen activated protein kinase (MAPK) pathway, the phosphatidylinosital-3 kinase (PI3K)-Akt pathway, the stress-activated protein kinase (SAPK) pathway, and the signal transducers and activators of transcription (STAT) pathways. Non-limiting examples of proteins involved in such pathways (and to which a small molecule EGFR antagonist according to the present invention can bind) include GRB-2, SOS, Ras, Raf, MEK, MAPK, and matrix metalloproteinases (MMPs).

One example of a small molecule EGFR antagonist is IRESSA™ (ZD1939), which is a quinozaline derivative that functions as an ATP-mimetic to inhibit EGFR. See U.S. Pat. No. 5,616,582 (Zeneca Limited); WO 96/33980 (Zeneca Limited) at p. 4; see also, Rowinsky et al., Abstract 5 presented at the 37th Annual Meeting of ASCO, San Francisco, Calif., 12-15 May 2001; Anido et al., Abstract 1712 presented at the 37th Annual Meeting of ASCO, San Francisco, Calif., 12-15 May 2001. Another example of a small molecule EGFR antagonist is TARCEVA™ (OSI-774), which is a 4-(substitutedphenylamino)quinozaline derivative [6,7-Bis(2-methoxy-ethoxy)-quinazolin-4-yl]-(3-ethynyl-phenyl)amine hydrochloride] EGFR inhibitor. See WO 96/30347 (Pfizer Inc.) at, for example, page 2, line 12 through page 4, line 34 and page 19, lines 14-17. See also Moyer et al., Cancer Res., 57: 4838-48 (1997); Pollack et al., J. Pharmacol., 291: 739-48 (1999). TARCEVA™ may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., 12-15 May 2001.

Other small molecules are also reported to inhibit EGFR, many of which are thought to being to the tyrosine kinase domain of an EGFR. Some examples of such small molecule EGFR antagonists are described in WO 91/116051, WO 96/30347, WO 96/33980, WO 97/27199 (Zeneca Limited), WO 97/30034 (Zeneca Limited), WO 97/42187 (Zeneca Limited), WO 97/49688 (Pfizer Inc.), WO 98/33798 (Warner Lambert Company), WO 00/18761 (American Cyanamid Company), and WO 00/31048 (Warner Lambert Company). Examples of specific small molecule EGFR antagonists include CI-1033 (Pfizer), which is a quinozaline (N-[4-(3-chloro-4-fluoro-phenylamino)-7-(3-morpholin-4-yl-propoxy)-quinazolin-6-yl]-acrylamide) inhibitor of tyrosine kinases, particularly EGFR and is described in WO 00/31048 at page 8, lines 22-6; PKI166 (Novartis), which is a pyrrolopyrimidine inhibitor of EGFR and is described in WO 97/27199 at pages 10-12; GW2016 (GlaxoSmithKline), which is an inhibitor of EGFR and HER2; EKB569 (Wyeth), which is reported to inhibit the growth of tumor cells that overexpress EGFR or HER2 in vitro and in vivo; AG-1478 (Tryphostin), which is a quinazoline small molecule that inhibits signaling from both EGFR and erbB-2; AG-1478 (Sugen), which is bisubstrate inhibitor that also inhibits protein kinase CK2; PD 153035 (Parke-Davis) which is reported to inhibit EGFR kinase activity and tumor growth, induce apoptosis in cells in culture, and enhance the cytotoxicity of cytotoxic chemotherapeutic agents; SPM-924 (Schwarz Pharma), which is a tyrosine kinase inhibitor targeted for treatment of prostrate cancer; CP-546,989 (OSI Pharmaceuticals), which is reportedly an inhibitor of angiogenesis for treatment of solid tumors; ADL-681, which is a EGFR kinase inhibitor targeted for treatment of cancer; PD 158780, which is a pyridopyrimidine that is reported to inhibit the tumor growth rate of A4431 xenografts in mice; CP-358,774, which is a quinazoline that is reported to inhibit autophosphorylation in HN5 xenografts in mice; ZD1839, which is a quinzoline that is reported to have antitumor activity in mouse xenograft models including vulvar, NSCLC, prostrate, ovarian, and colorectal cancers; CGP 59326A, which is a pyrrolopyrimidine that is reported to inhibit growth of EGFR-positive xenografts in mice; PD 165557 (Pfizer); CGP54211 and CGP53353 (Novartis), which are dianilnophthalimides. Naturally derived EGFR tyrosine kinase inhibitors include genistein, herbimycin A, quercetin, and erbstatin.

Further small molecules reported to inhibit EGFR and that are therefore within the scope of the present invention are tricyclic compounds such as the compounds described in U.S. Pat. No. 5,679,683; quinazoline derivatives such as the derivatives described in U.S. Pat. No. 5,616,582; and indole compounds such as the compounds described in U.S. Pat. No. 5,196,446.

In another embodiment, the EGFR antagonist can be administered in combination with one or more suitable adjuvants, such as, for example, cytokines (IL-10 and IL-13, for example) or other immune stimulators, such as, but not limited to, chemokine, tumor-associated antigens, and peptides. See, e.g., Larrivée et al., supra. It should be appreciated, however, that administration of only a antibody 11F8 mimetic is sufficient to prevent, inhibit, or reduce the progression of the tumor in a therapeutically effective manner.

For combination therapies, the antibody 11F8 mimetic and anti-neoplastic agent or receptor antagonist may be administered concomitantly or sequentially.

This invention also provides a pharmaceutical composition/formulation containing a antibody 11F8 mimetic and a pharmaceutically acceptable carrier. Carrier as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. In one variation, at least one non-aqueous carrier is used. Examples of physiologically acceptable carriers include buffers such as phosphate, citrate and other organic acids; antioxidants including ascorbic acid; low molecular weight (less than about 10 residues) polypeptide; proteins, such as serum albumin, gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such, as TWEEN®, polyethylene glycol (PEG), and PLURONICS®.

The active ingredients may also be entrapped in microcapsules prepared, for example, by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes. Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The present invention also includes kits for inhibiting tumor growth and/or tumor-associated angiogenesis comprising a therapeutically effective amount of a antibody 11F8 mimetic. The kits can further contain any suitable antagonist of, for example, another growth factor receptor involved in tumorigenesis or angiogenesis (e.g., VEGFR-1/Flt-1, VEGFR-2, PDGFR, IGFR, NGFR, FGFR, etc, as described above). Alternatively, or in addition, the kits of the present invention can further comprise an anti-neoplastic agent. Examples of suitable anti-neoplastic agents in the context of the present invention have been described herein. The kits of the present invention can further comprise an adjuvant; examples have also been described above.

Moreover, included within the scope of the present invention is use of the present antibodies in vivo and in vitro for investigative or diagnostic methods, which are well known in the art. The diagnostic methods include kits, which contain mimetics of the present invention.

Accordingly, the mimetics can be used in vivo and in vitro for investigative, diagnostic, prophylactic, or treatment methods, which are well known in the art. Of course, it is to be understood and expected that variations in the principles of invention herein disclosed can be made by one skilled in the art and it is intended that such modifications are to be included within the scope of the present invention.

All patents and other documents cited herein are incorporated by reference in their entireties.

### Examples

The following examples are offered for illustrative purposes only, and are not intended to limit the scope of the present invention in any way.

Protein expression and purification. The soluble extracellular domain of EGFR (sEGFR) and the isolated domain III of sEGFR (sEGFRd3) were produced exactly as described (Ferguson et al., 2000; Li et al., 2005), and were used without modification of their glycosylation state. Each of sEGFR and sEGFRd3 were further purified by size exclusion chromatography (SEC) using a SEC250 column (BioRad) pre-equilibrated with 25 mM HEPES, 100 mM NaCl, pH 7.5 and concentrated to 6.2 mg/ml. antibody 11F8 Fab fragment was prepared by treatment of the IgG protein with papain. The IgG protein (20 mg/ml) was incubated with papain (1:1000 w:w) at 37° C. for one hour and the digestion was terminated by addition of iodoacetemide (75 mM final concentration). The reaction mixture was loaded onto a Protein-A column and the flow-through fraction containing the Fab fragments was collected and concentrated. The antibody 11F8Fab was fractionated by SEC and mixed with sEGFR to give a two fold molar excess of Fab over sEGFR. Excess Fab was separated from the sEGFR:Fab complex using the same SEC column. The peak fractions containing the sEGFR:Fab complex (as confirmed by SDS-PAGE), were concentrated to 11 mg/ml. Purified complexes were concentrated and buffer exchanged into 25 mM HEPES, pH 7.5, containing 50 mM NaCl.

Crystallization and data collection. Fab11F8/sEGFR and Fab11F8/sEGFRd3 complexes were crystallized by the hanging drop vapor diffusion method. For Fab11F8/sEGFR drops containing equal parts of Fab11F8/sEGFR complex (10 mg/ml) and of a reservoir solution of 12% PEG 3350, 1 M NaCl, 50 mM MES (pH 6.5) were equilibrated over this reservoir at 25° C. Small single crystals appeared after several days. To prevent additional nucleation and to promote the growth of large (0.25×0.1×0.02 mm) single crystals, the crystallization trays were sequentially moved to conditions of decreasing temperature over two weeks, to a final temperature of 4° C. Fab11F8/sEGFRd3 complex crystals were obtained by mixing equal parts of Fab11F8/sEGFRd3 complex solution (6 mg/ml) with a reservoir solution of 12% PEG 3350, 250 mM ammonium sulfate, 50 mM sodium acetate (pH 5.0) and equilibrating this over reservoir of this same solution at 25° C. Streak seeding was used to produce large (0.15×0.15×0.05 mm) single crystals. In each case crystals were briefly exposed to a cryostabilizer of reservoir solution supplemented with 15% ethylene glycol and were flash frozen in liquid nitrogen.

X-ray diffraction data were collected at the Cornell High Energy Synchrotron Source (CHESS) beamlines A1 (Fab11F8/sEGFR) and F2 (Fab11F8/sEGFRd3), using an ADSC Quantum-210 CCD detector. All data were processed using HKL2000 (Otwinowski and Minor, 1997). Data collection statistics are given in Table 1. The small deviation of the beta angle from 90° (90.02° initially led us to assign the Fab11F8/sEGFRd3 crystal to an orthorhombic point group. The data could not be merged in this higher symmetry leading to high R-factors and a very large number of reflections flagged for rejection.

Structure determination and refinement. Molecular replacement (MR) methods were used to solve both structures. In each case search models for sEGFR were derived from the coordinates of the FabC225/sEGFR complex (pdb id 1yy9) while for Fab11F8 a homology search model was generated using the program MODELLER (Eswar et al., 2006). The template for this Fab model comprised the light chain from the Fab fragment of the human IgM cold agglutinin (pdb id 1dn0) and heavy chain of the CAMPATH-1H Fab fragment (pdb id 1ce1). For Fab/sEGFR an initial solution was found for the Fab plus domain III (amino acids 310-501) of sEGFR using the program MOLREP (CCP4, 1994; Vagin and Teplyakov, 1997). Attempts to locate domains I, II and IV using MR methods were unsuccessful. Following rounds of manual model building in O (Jones et al., 1991) and refinement combined with density modification using the programs REFMAC (Murshudov et al., 1997) and DM (CCP4, 1994), interpretable density was seen for domain IV and part of domain II of sEGFR. No interpretable density could be seen for domain I. The current model comprising amino acids 239-614 of sEGFR plus the Fab11F8 packs to form disconnected layers. Domain I must be present to make crystal packing contacts in the third direction but is presumably statically disordered.

For Fab11F8/sEGFRd3, the initial MR search employed two model fragments: domain III of sEGFR and the Fv region of the Fab homology model. Eight copies of each fragment were located using automatic search protocols in the program PHASER (McCoy et al., 2005; Storoni et al., 2004). With the positions of these 8 Fv plus sEGFRd3 fragments fixed, the 8 Fc domains of the Fab could be located. The noncrystallographic symmetry (NCS) relationship between the 8 Fv/sEGFRd3 fragments and the Fc fragments differs slightly. Initially, 8-fold NCS averaging was applied to generate electronic density map using the program DM (CCP4, 1994) and the model was rebuilt using the program Coot (Emsley and Cowtan, 2004). In the later stages of refinement the NCS restrains were released. Refinement was carried out with REFMAC (CCP4, 1994). Refinement statistics are summarized in Table 1. All structure figures were prepared using PyMOL (DeLano, 2004).

BIAcore binding studies. Surface plasmon resonance binding experiments, performed using a BIAcore 3000 instrument, were performed in 10 mM Hepes buffer, pH 8.0, that contained 150 mM NaCl, 3 mM EDTA, and 0.005% Tween 20 (HBS-EP8) at 25° C. Fab11F8 (at 50 μg/ml in 10 mM sodium acetate at pH 5.5) was amine coupled to a CM5 BIAcore sensor chip and surface plasmon resonance (SPR) used to measure binding of wild type and mutated versions of sEGFR to this immobilized Fab11F8 exactly as described (Li et al., 2005). The effect of added Fab11F8 upon the binding of 600 nM sEGFR to immobilized EGF was determined as described (Li et al., 2005). Data were analyzed using Prism 4 (GraphPad Software, Inc.).

Generation of binding site sEGFR mutations. Standard PCR directed mutagenesis strategies were used to produce the appropriate DNA in the pFastBac vector. Targeted residues were mutated to alanine with the exception of S468 that was mutated to an isoleucine to introduce a larger side chain. An S468I mutation has been reported to disrupt binding of another antibody that binds to domain III (mAb 13A9)(Chao et al., 2004). The following mutations were made: Q408A/Q409A, Q384A/Q408A/Q409A, K443A, S4681, N473A, S468I/N473A. The generation of baculovirus, overexpression and purification of the altered forms of sEGFR was performed exactly as reported for wild type sEGFR (Ferguson et al., 2000).

EGFR:11F8 Fab Interface. The following amino acids are involved in direct hydrogen bonds with the Fab (3.25 Å cut-off, calculated using the program CONTACT (CCP4)):

| | 11F8* | 11F8* | |
|---|---|---|---|
| sEGFR | Light Chain | Heavy Chain | Type |
| Thr 464 | Tyr 32 | | Main chain-side chain |
| Ile 466 | Tyr 32 | | Main chain-side chain |
| Ser 468 | Tyr 91 | | Side chain-main chain |
| Asn 469 | Thr 94 | | Side chain-main chain |
| Gln 384 | | Tyr 55 | Side chain-side chain |
| His 409 | | Asp 33 | Side chain-side chain |
| Ser 418 | | Trp 52 | Side chain-side chain |
| Ser 440 | | Tyr 35 | Side chain-main chain |
| Gly 441 | | Tyr 52 | Main chain-side chain |
| Lys 443 | | Thr 59 | Side chain-main chain |

*amino acids in the Fab are numbered in a simple sequential manner.

Additional amino acids that are close (4 Å cut-off) are shown on the following sequence.

310 320 330 340
B1 RKVCNGIGIG EFKDSLSINA TNIKHFKNCT SISGDLHILP
350 360 370 380
VAFRGDSFTH TPPLDPQELD ILKTVKEITG FLLIQAWPEN
390 400 410 420
RTDLHAFENL EIIRGRTKQH GQFSLAVVSL NITSLGLRSL
430 440 450 460
KEISDGDVII SGNKNLCYAN TINWKKLFGT SGQKTKIISN
470
RGENSCKA

| | |
|---|---|
| Bold | Fab Heavy chain |
| Underlined and Italic | Fab Light chain |
| Italic | Both chains of Fab |

The binding site for 11F8 Fab is partially over-lapping with the ligand binding site. The following amino acids are involved in contact to TGF-α or EGF, as reported by Garrett et al. and Ogiso et al.

310 320 330 340
B1 RKVCNGIGIG EFKDSLSINA TNIKHFKNCT SISGDLHILP
350 360 370 380
VAFRGDSFTH TPPLDPQELD ILKTVKEITG FLLIQAWPEN
390 400 410 420
RTDLHAFENL EIIRGRTKQH GQFSLAVVSL NITSLGLRSL
430 440 450 460
KEISDGDVII SGNKNLCYAN TINWKKLFGT SGQKTKIISN
470
RGENSCKA

| | |
|---|---|
| Underlined | EGF/TGF-α |

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above, without departing from the spirit or scope of the invention.

### REFERENCES

1. Al-Lazikani, B., Lesk, A. M., and Chothia, C. (1997). Standard conformations for the canonical structures of immunoglobulins. J. Mol. Biol. 273, 927-948.
2. Baker, H. M., Day, C. L., Norris, G. E., and Baker, E. N. (1994). Enzymatic deglycosylation as a tool for crystallization of mammalian binding proteins. Acta crystallographica 50, 380-384.
3. Baker, N. A., September, D., Joseph, S., Holst, M. J., and McCammon, J. A. (2001). Electrostatics of nanosystems: application to microtubules and the ribosome. Proc. Natl. Acad. Sci. USA 98, 10037-10041.
4. Bouyain, S., Longo, P. A., Li, S., Ferguson, K. M., and Leahy, D. J. (2005). The extracellular region of ErbB4 adopts a tethered conformation in the absence of ligand. Proc. Natl. Acad. Sci. U.S.A. 102, 15024-15029.
5. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998). Crystallography & NMR system: A new software suite for macromolecular structure determination. Acta Crystallogr D Biol Crystallogr 54 (Pt 5), 905-921.
6. Burgess, A. W., Cho, H. S., Eigenbrot, C., Ferguson, K. M., Garrett, T. P., Leahy, D. J., Lemmon, M. A., Sliwkowski, M. X., Ward, C. W., and Yokoyama, S. (2003). An open-and-shut case? Recent insights into the activation of EGF/ErbB receptors. Molecular cell 12, 541-552.
7. CCP4 (1994). The CCP4 Suite: Programs for Protein Crystallography. Acth Cryst. D50, 760-763. Chao, G., Cochran, J. R., and Wittrup, K. D. (2004). Fine epitope mapping of anti-epidermal growth factor receptor antibodies through random mutagenesis and yeast surface display. J. Mol. Biol. 342, 539-550.
8. Cho, H. S., and Leahy, D. J. (2002). Structure of the extracellular region of HERS reveals an interdomain tether. Science 297, 1330-1333.
9. Cho, H. S., Mason, K., Ramyar, K. X., Stanley, A. M., Gabelli, S. B., Denney, D. W., Jr., and Leahy, D. J. (2003). Structure of the extracellular region of HER2 alone and in complex with the Herceptin Fab. Nature 421, 756-760.
10. Chothia, C., and Lesk, A. M. (1987). Canonical structures for the hypervariable regions of immunoglobulins. J. Mol. Biol. 196, 901-917.
11. Chothia, C., Lesk, A. M., Levitt, M., Amit, A. G., Mariuzza, R. A., Phillips, S. E., and Poljak, R. J. (1986). The predicted structure of immunoglobulin D1.3 and its comparison with the crystal structure. Science 233, 755-758.
12. Clackson, T., Ultsch, M. H., Wells, J. A., and de Vos, A. M. (1998). Structural and functional analysis of the 1:1 growth hormone:receptor complex reveals the molecular basis for receptor affinity. J. Mol. Biol. 277, 1111-1128.
13. Clark, L. A., Boriack-Sjodin, P. A., Eldredge, J., Fitch, C., Friedman, B., Hanf, K J., Jarpe, M., Liparoto, S. F., Li, Y., Lugovskoy, A., et al. (2006). Affinity enhancement of an in vivo matured therapeutic antibody using structure-based computational design. Protein Sci. 15, 949-960.
14. Dawson, J. P., Berger, M. B., Lin, C. C., Schlessinger, J., Lemmon, M. A., and Ferguson, K. M. (2005). Epidermal growth factor receptor dimerization and activation require ligand-induced conformational changes in the dimer interface. Mol. Cell. Biol. 25, 7734-7742.
15. Dawson, J. P., Bu, Z., and Lemmon, M. A. (2007). Ligand-induced structural transitions in ErbB receptor extracellular domains. Structure, in press.
16. de Haard, H. J., van Neer, N., Reurs, A., Hufton, S. E., Roovers, R. C., Henderikx, P., de Bruine, A. P., Arends, J. W., and Hoogenboom, H. R. (1999). A large non-immunized human Fab fragment phage library that permits rapid isolation and kinetic analysis of high affinity antibodies. J. Biol. Chem. 274, 18218-18230.
17. DeLano, W. L. (2004). The PyMOL Molecular Graphics System. (Palo Alto, Calif., USA., DeLano Scientific).
18. Emsley, P., and Cowtan, K. (2004). Coot: model-building tools for molecular graphics. Acta crystallographica 60, 2126-2132.
19. Eswar, N., Webb, B., Marti-Renom, M. A., Madhusudhan, M. S., Eramian, D., Shen, M., Pieper, U., and Sali, A. (2006). Comparative Protein Structure Modeling Using MODELLER., Vol Supplement 15, 5.6.1-5.6.30 (New York, N.Y.: John Wiley & Sons, Inc.)
20. Fellouse, F. A., Li, B., Compaan, D. M., Peden, A. A., Hymowitz, S. G., and Sidhu, S. S. (2005). Molecular recognition by a binary code. J. Mol. Biol. 348, 1153-1162
21. Ferguson, K. M. (2004). Active and inactive conformations of the epidermal growth factor receptor. Biochem. Soc. Trans. 32, 742-745.
22. Ferguson, K. M., Berger, M. B., Mendrola, J. M., Cho, H. S., Leahy, D. J., and Lemmon, M. A. (2003). EGF activates its receptor by removing interactions that autoinhibit ectodomain dimerization. Molecular Cell 11, 507-517.
23. Ferguson, K. M., Darling, P. J., Mohan, M. J., Macatee, T. L., and Lemmon, M. A. (2000). Extracellular domains drive homo- but not hetero-dimerization of erbB receptors. EMBO J. 19, 4632-4643.
24. Franklin, M. C., Carey, K. D., Vajdos, F. F., Leahy, D. J., de Vos, A. M., and Sliwkowski, M. X. (2004). Insights into ErbB signaling from the structure of the ErbB2-pertuzumab complex. Cancer cell 5, 317-328.
25. French, A. R., Tadaki, D. K., Niyogi, S. K., and Lauffenburger, D. A. (1995). Intracellular trafficking of epidermal growth factor family ligands is directly influenced by the pH sensitivity of the receptor/ligand interaction. J. Biol. Chem. 270, 4334-4340.
26. Garrett, T. P., McKem, N. M., Lou, M., Elleman, T. C., Adams, T. E., Lovrecz, G. O., Kofler, M., Jorissen, R. N., Nice, E. C., Burgess, A. W., and Ward, C. W. (2003). The crystal structure of a truncated ErbB2 ectodomain reveals an active conformation, poised to interact with other ErbB receptors. Molecular cell 11, 495-505.
27. Garrett, T. P., McKern, N. M., Lou, M., Elleman, T. C., Adams, T. E., Lovrecz, G. O., Zhu, H. J., Walker, F., Frenkel, M. J., Hoyne, P. A., et al. (2002). Crystal structure of a truncated epidermal growth factor receptor extracellular domain bound to transforming growth factor alpha. Cell 110, 763-773.
28. Grueninger-Leitch, F., D'Arcy, A., D'Arcy, B., and Chene, C. (1996). Deglycosylation of proteins for crystallization using recombinant fusion protein glycosidases. Protein Sci. 5, 2617-2622.
29. Johns, T. G., Adams, T. E., Cochran, J. R., Hall, N. E., Hoyne, P. A., Olsen, M. J., Kim, Y. S., Rothacker, J., Nice, E. C., Walker, F., et al. (2004). Identification of the epitope for the epidermal growth factor receptor-specific monoclonal antibody 806 reveals that it preferentially recognizes an untethered form of the receptor. J. Biol. Chem. 279, 30375-30384.
30. Jones, T. A., Zou, J. Y., Cowan, S. W., and Kjeldgaard (1991). Improved methods for building protein models in electron density maps and the location of errors in these models. Acta Crystallogr. A 47 (Pt 2), 110-119.
31. Jorgensen W. L., (2004). The many roles of computation on drug discovery. Science 303, 1813-1818.
32. Kohda, D., Odaka, M., Lax, I., Kawasaki, H., Suzuki, K., Ullrich, A., Schlessinger, J., and Inagaki, F. (1993). A 40-kDa epidermal growth factor/transforming growth factor alpha-binding domain produced by limited proteolysis of the extracellular domain of the epidermal growth factor receptor. J. Biol. Chem. 268, 1976-1981.
33. Kuenen, B., Witteveen, E., Ruijter, R., Ervin-Haynes, A., Tjin-A-ton, M., Fox, F., Ding, C., Giaccone, G., and Voest, E. E. (2006). A phase I study of antibody 11F8, a fully human anti-epidermal growth factor receptor (EGFR) IgG1 monoclonal antibody in patients with solid tumors. Interim results. J. Clin. Oncol., 2006 ASCO Annual Meeting Proceedings Part I., 24, 3024.
34. Landau, M., Mayrose, I., Rosenberg, Y., Glaser, F., Martz, E., Pupko, T., and Ben-Tal, N. (2005). ConSurf 2005: the projection of evolutionary conservation scores of residues on protein structures. Nuc. Acids Res. 33, W299-302.
35. Lawrence, M. C., and Colman, P. M. (1993). Shape complementarity at protein/protein interfaces. J. Mol. Biol. 234, 946-950.
36. Lemmon, M. A., Bu, Z., Ladbury, J. E., Zhou, M., Pinchasi, D., Lax, I., Engelman, D. M., and Schlessinger, J. (1997). Two EGF molecules contribute additively to stabilization of the EGFR dimer. EMBO J. 16, 281-294.
37. Lenz, H. J. (2007). Management and preparedness for infusion and hypersensitivity reactions. The oncologist 12, 601-609.
38. Li, S., Schmitz, K. R., Jeffrey, P. D., Wiltzius, J. J., Kussie, P., and Ferguson, K. M. (2005). Structural basis for inhibition of the epidermal growth factor receptor by cetuximab. Cancer cell 7, 301-311.
39. Liu, M., Zhang, H., Jimenez, X., Ludwig, D. L., Witte, L., Bohlen, P., Hicklin, D. J., and Zhu, Z. (2004). Identification and characterization of a fully human antibody directed against epidermal growth factor receptor for cancer therapy. AACR Meeting Abstracts 2004, 163-c.
40. Lu, D., Jimenez, X., Witte, L., and Zhu, Z. (2004a). The effect of variable domain orientation and arrangement on the antigen-binding activity of a recombinant human bispecific diabody. Biochem. Biophys. Res. Commun. 318, 507-513.
41. Lu, D., Zhang, H., Koo, H., Tonra, J., Balderes, P., Prewett, M., Corcoran, E., Mangalampalli, V., Bassi, R., Anselma, D., et al. (2005). A fully human recombinant IgG-like bispecific antibody to both the epidermal growth factor receptor and the insulin-like growth factor receptor for enhanced antitumor activity. J. Biol. Chem. 280, 19665-19672.
42. Lu, D., Zhang, H., Ludwig, D., Persaud, A., Jimenez, X., Burtrum, D., Balderes, P., Liu, M., Bohlen, P., Witte, L., and Zhu, Z. (2004b). Simultaneous blockade of both the epidermal growth factor receptor and the insulin-like growth factor receptor signaling pathways in cancer cells with a fully human recombinant bispecific antibody. J. Biol. Chem. 279, 2856-2865.
43. Marshall, J. (2006). Clinical implications of the mechanism of epidermal growth factor receptor inhibitors. Cancer 107, 1207-1218.
44. McCoy, A. J. (2007). Solving structures of protein complexes by molecular replacement with Phaser. Acta crystallographica 63, 32-41.
45. McCoy, A. J., Grosse-Kunstleve, R. W., Storoni, L. C., and Read, R. J. (2005). Likelihood-enhanced fast translation functions. Acta crystallographica 61, 458-464.
46. Murshudov, G. N., Vagin, A. A., and Dodson, E. J. (1997). Refinement of macromolecular structures by the maximum-likelihood method. Acta crystallographica 53, 240-255.
47. Nunez, M., Mayo, K. H., Starbuck, C., and Lauffenburger, D. (1993). pH sensitivity of epidermal growth factor receptor complexes. J. Cell. Biochem. 51, 312-321.
48. Ogiso, H., Ishitani, R., Nureki, 0., Fukai, S., Yamanaka, M., Kim, J. H., Saito, K., Sakamoto, A., Inoue, M., Shirouzu, M., and Yokoyama, S. (2002). Crystal structure of the complex of human epidermal growth factor and receptor extracellular domains. Cell 110, 775-787.
49. Otwinowski, Z., and Minor, W. (1997). Processing of X-ray Diffraction Data Collected in Oscillation Mode. In Macromolecular Crystallography, C. W. Carter Jr., and R. M. Sweet, eds. (New York: Academic Press), pp. 307-326.
50. Prewett, M., Tonra, J. R., Rajiv, B., Hooper, A. T., Makhoul, G., Finnerty, B., Witte, L., Bohlen, P., Zhu, Z., and Hicklin, D. J. (2004). Antitumor activity of a novel, human anti-epidermal growth factor receptor (EGFR) monoclonal antibody (antibody 11F8) in human tumor xenograft models. AACR Meeting Abstracts 2004, 1235.
51. Scaltriti, M., and Baselga, J. (2006). The epidermal growth factor receptor pathway: a model for targeted therapy. Clin. Cancer Res. 12, 5268-5272.
52. Schlessinger, J. (2000). Cell signaling by receptor tyrosine kinases. Cell 103, 211-225. Stanfield, R. L., Zemla, A., Wilson, I. A., and Rupp, B. (2006). Antibody elbow angles are influenced by their light chain class. J. Mol. Biol. 357, 1566-1574.
53. Storoni, L. C., McCoy, A. J., and Read, R. J. (2004). Likelihood-enhanced fast rotation functions. Acta crystallographica 60, 432-438.
54. Sunada, H., Magun, B. E., Mendelsohn, J., and MacLeod, C. L. (1986). Monoclonal antibody against epidermal growth factor receptor is internalized without stimulating receptor phosphorylation. Proc. Natl. Acad. Sci. U.S.A. 83, 3825-3829.
55. Todaro, G. J., De Larco, J. E., and Cohen, S. (1976). Transformation by murine and feline sarcoma viruses specifically blocks binding of epidermal growth factor to cells. Nature 264, 26-31
56. Vagin, A., and Teplyakov, A. (1997). MOLREP: an Automated Program for Molecular Replacement. J. Appl. Crystallogr. 30, 1022-1025.
57. Waterman, H., Sabanai, I., Geiger, B., and Yarden, Y. (1998). Alternative intracellular routing of ErbB receptors may determine signaling potency. J. Biol. Chem. 273, 13819-13827.
58. Weiner, L. M. (2006). Fully human therapeutic monoclonal antibodies. J. Immunother. 29, 1-9. Zhang, X., Gureasko, J., Shen, K., Cole, P. A., and Kuriyan, J. (2006). An allosteric mechanism for activation of the kinase domain of epidermal growth factor receptor. Cell 125, 1137-1149.
59. Winn, M. D., Isupov, M. N., and Murshudov, G. N. (2001). Use of TLS parameters to model anisotropic displacements in macromolecular refinement. Acta Crystallogr D Biol Crystallogr 57, 122-133.
60. Zhen, Y., Caprioli, R. M., and Staros, J. V. (2003). Characterization of glycosylation sites of the epidermal growth factor receptor. Biochemistry 42, 5478-5492.

TABLE 1
Data collection and refinement statistics

| | Fab11F8/sEGFRd3 complex | Fab11F8/sEGFR complex |
|---|---|---|
| Data Collection Statistics^{a} | | |
| Space group | P2₁ | C222₁ |
| Unique cell dimensions | a = 154.4 Å, b = 139.1 Å, c = 175.3 Å; β = 90.02° | a = 77.8 Å, b = 70.9 Å, c = 147.1 Å |
| X-ray source | CHESS F2 | CHESS A1 |
| Resolution limit | 2.6 Å | 3.3 Å |
| Observed/unique | 875,685/231,396 | 132,555/21,995 |
| Redundancy | 3.8 fold | 6 fold |
| Completeness (%) | 95.6 (96.8) | 100.0 (100.0) |
| R_{sym}^{b} | 0.097 (0.456) | 0.188 (0.580) |
| <I/σ> | 13.1 (2.4) | 9.13 (3.1) |
| Refinement Statistics | | |
| Resolution limits | 43-2.6 Å | 50-3.3 Å |
| No. of reflections/no. test set | 231,396/11,664 | 22,470/1,206 |
| R factor (R_{free})^{c} | 0.23 (0.29) | 0.28 (0.35) |
| Model | | |
| Protein | 8 Fab11F8/sEGFRd3 complexes | 1 Fab11F8/sEGFR complex |
| | aa 310-502 (sEGFRd3)^{d} | aa 239-617 (sEGFR) |
| | aa 1-213 (light chain) | aa 1-213 (light chain) |
| | aa 1-222 (heavy chain); missing aa 137-141^{e} | aa 2-221 (heavy chain) |
| | 64 saccharide units | 10 saccharide units |
| | 577 water molecules | |
| Total number of atoms | 38,850 | 6,054 |
| RMSD bond lengths (Å) | 0.011 | 0.022 |
| RMSD bond angles (°) | 1.43 | 2.47 |

Legend of Table 1:
^{a}Numbers in parentheses refer to last resolution shell
^{b}R_{sym} = Σ|Iₕ − <Iₕ>|/ΣIₕ, where <Iₕ> = average intensity over symmetry equivalent measurements
^{c}R factor = Σ|Fₒ − F_{c}|/ΣFₒ, where summation is over data used in the refinement; R_{free} includes only 5% of the data excluded from the refinement
^{d}Chains A and M start at 308, chains B, E and S start at 309.
^{e}Number of missing amino acids varies by chain, maximum of 8 aa missing (chain C).

What is claimed:

1. A crystal of a receptor-antibody complex comprising a receptor-antibody complex of soluble epidermal growth factor receptor extracellular domain (sEGFR) or isolated extracellular domain 3 of EGFR (EGFRd3) and 11F8 Fab, wherein the crystal has a resolution determined by X-ray crystallography of better than a value selected from the group consisting of about 5.0 Angstroms, about 4.0 Angstroms, and about 3.0 Angstroms.

2. (canceled)

3. (canceled)

4. The crystal of claim 1, wherein the crystal is of soluble EGFR and 11F8 Fab and wherein the crystal belongs to space group C222₁ and has unit cell dimensions a=77.8 Å, b=70.9 Å, and c=147.1 Å.

5. The crystal of claim 1, wherein the crystal is of EGFRd3 and 11F8 Fab and wherein the crystal belongs to space group P2₁, and has unit cell dimensions a=154.4 Å, b=139.1 Å, c=175.3 Å; β=90.02°.

6. The crystal of claim 1, wherein the crystal is of EGFRd3 and 11F8 Fab and has the atomic coordinates provided in Table 2.

7. The crystal of claim 1, wherein the crystal is of soluble EGFR and 11F8 Fab and has the atomic coordinates provided in Table 3.

8. A method for preparing a crystal of a complex of an epidermal growth factor receptor (EGFR) extracellular domain and antibody 11F8 Fab comprising the steps of:
preparing a solution containing (i) soluble EGFR or isolated domain III of EGFR and (ii) antibody 11F8 Fab fragment; and
growing the crystal.

9. The method of claim 6, wherein the pH of the solution is about 6.0 to about 8.0.

10. A method of identifying a mimetic of antibody 11F8 comprising comparing a three-dimensional structure of the mimetic with a three-dimensional structure determined for the complex of claim 1.

11. The method of claim 10, wherein the three dimensional structure of the mimetic is compared with at least a subset of the coordinates provided in Table 2 or Table 3.

12. The method of claim 10, wherein identifying a mimetic is carried out by comparing the three-dimensional structure of the mimetic against the coordinates of at least one EGFR amino acid bound by antibody 11F8 Fab.

13. The method of claim 12, wherein the EGFR amino acid is selected from the group consisting of Pro349, Gln384, His409, Ser418, Ile438, Ser440, Gly441, Lys443, Thr464, Lys465, Thr466, Ile467, Ser468, Asn469, Gly471, and Asn473.

14. The method of claim 10, wherein the locations of atoms of the mimetic that contact EGFR correspond to atoms of antibody 11F8 that contact EGFR.

15. The method of claim 10, wherein identifying a mimetic comprises comparing a three dimensional structure of a mimetic with the atomic coordinates of a region of EGFR selected from the group consisting of about amino acid residue 348 to about amino acid residue 354, about amino acid residue 380 to about amino acid residue 385, about amino acid residue 405 to about amino acid residue 420, about amino acid residue 435 to about amino acid residue 475 and combinations thereof.

16. The method of claim 10, wherein the mimetic is selected from the group consisting of a small molecule and a peptide.

17. (canceled)

18. The method of claim 16, wherein the peptide is an antibody or a fragment thereof.

19. The method of claim 10, wherein the method is carried out with use of a computer.

20. The method of claim 10, further comprising synthesizing the mimetic and assaying its binding or physiological activity.

21. The method of claim 10, wherein the mimetic has a property selected, from the group consisting of binds to EGFR with similar affinity as antibody 11F8 Fab, inhibits dimerization of EGFR expressed by a cell, inhibits tyrosine kinase activity of the receptor, and blocks binding of EGF to EGFR.

22. (canceled)

23. (canceled)

24. (canceled)

25. A method of identifying a mimetic of antibody 11F8, comprising:
(a) introducing in silico substitutions in at least a single CDR region of antibody 11F8 to obtain a pool of variants; and
(b) using a computer and at least a subset of the EGFR coordinates provided in Table 2 or Table 3 to select a variant with improved EGFR binding characteristics wherein the variant so selected is said mimetic.

26. The method of claim 25, further comprising determining the biological activity of the mimetic.

27. The method of claim 25, wherein at most a single substitution is made in each CDR.

28. The method of claim 25, wherein substitutions are made solely in a CDR3 region.

29. A computer-assisted method for identifying a potential antagonist mimetic that binds the extracellular domain of EGFR comprising a processor, a data storage system, an input device, and an output device, comprising:
(a) inputting into the programmed computer through said input device data comprising the three-dimensional coordinates of a subset of the atoms of EGFR as set out in Table 2 or Table 3;
(b) providing a database of chemical and peptide structures stored in said computer data storage system;
(c) selecting from said database, using computer methods, structures having a portion that is structurally similar to said criteria data set; and
(d) outputting to said output device the selected chemical structures having a portion similar to said criteria data set.

30. A machine-readable medium having stored thereon a plurality of executable instructions to perform a method of identifying a mimetic of antibody 11F8 using a crystal of a receptor-antibody complex comprising a receptor-antibody complex of an epidermal growth factor receptor (EGFR) extracellular domain and antibody 11F8 Fab, the method comprising: comparing a three-dimensional structure of a mimetic with a three dimensional structure an epidermal growth factor receptor (EGFR) extracellular domain and antibody 11F8 Fab having an X-ray crystallography resolution of better than about 5.0 Angstroms.

31. The machine-readable medium of claim 30, wherein the EGFR coordinates comprise at least a subset of the atomic coordinates of Table 2.

32. The machine-readable medium of claim 30, wherein the three-dimensional structure of the mimetic is compared with at least a subset of the atomic coordinates of Table 2.

33. The machine-readable medium of claim 30, wherein identifying a mimetic comprises comparing the three-dimensional structure of a mimetic with a three-dimensional structure of at least one EGFR amino acid bound by antibody 11F8 Fab.

34. The machine-readable medium of claim 30, wherein identifying a mimetic comprises comparing a three dimensional structure of a mimetic with the atomic coordinates of a region of EGFR selected from the group consisting of about amino acid residue 348 to about amino acid residue 354, about amino acid residue 380 to about amino acid residue 385, about amino acid residue 405 to about amino acid residue 420, about amino acid residue 435 to about amino acid residue 475 and combinations thereof.

35. A machine-readable medium having stored thereon a plurality of executable instructions to perform a method for identifying a mimetic of antibody 11F8, the method comprising:
(a) introducing in silico substitutions in at least a single CDR region of antibody 11F8 to obtain a pool of variants; and
(b) using a computer and at least a subset of the EGFR coordinates provided in Table 2 or Table 3 to select a variant with improved EGFR binding characteristics.

36. An antibody 11F8 mimetic identified by the method of claim 25.

37. A method of inhibiting EGFR comprising administering a mimetic of claim 36.

38. A method of inhibiting tumor growth in a mammal comprising administering a therapeutically effective amount of an antibody 11F8 mimetic of claim 36, wherein said tumor has the property selected from the group consisting of expressing EGFR, overexpressing EGFR, is a primary tumor, is a metastatic tumor, is a refractory tumor, is a vascularized tumor, is a colorectal tumor, is a head and neck tumor, is a pancreatic tumor, is a lung tumor, is a breast tumor, is a renal cell carcinoma, and is a glioblastoma.

39. (canceled)

40. (canceled)

41. (canceled)

42. (canceled)

43. (canceled)

44. (canceled)

45. (canceled)

46. The method of claim 38, wherein the antibody 11F8 mimetic is administered in combination with an anti-neoplastic agent selected from the group consisting of, a chemotherapeutic agent, irinotecan (CPT-11), and radiation.

47. (canceled)

48. (canceled)

49. (canceled)

50. The method of claim 38, wherein the antibody 11F8 mimetic is administered in combination with an agent selected from the group consisting of an EGFR antagonist an intracellular EGFR antagonist, a VEGFR antagonist, and an insulin like growth factor receptor (IGFR) antagonist.

51. (canceled)

52. (canceled)

53. (canceled)

54. A method of treating a hyperproliferative disease comprising administering a therapeutically effective amount of an antibody 11F8 mimetic of claim 36.

55. The method of claim 54, wherein the hyperproliferative disease is psoriasis.

56. The method of claim 54, wherein the antibody 11F8 mimetic is administered in combination with a topical or systemic agent for psoriasis.

57. The method of claim 54, wherein the antibody 11F8 mimetic is administered in combination with an agent selected from the list consisting of a corticosteroid and a retinoid.

58. (canceled)

59. An antibody 11F8 mimetic identified by the method of claim 30.

60. A method of inhibiting EGFR comprising administering a mimetic of claim 59.

61. A method of inhibiting tumor growth in a mammal comprising administering a therapeutically effective amount of an antibody 11F8 mimetic of claim 30, wherein said tumor has the property selected from the group consisting of expressing EGFR, overexpressing EGFR, is a primary tumor, is a metastatic tumor, is a refractory tumor, is a vascularized tumor, is a colorectal tumor, is a head and neck tumor, is a pancreatic tumor, is a lung tumor, is a breast tumor, is a renal cell carcinoma, and is a glioblastoma.

62. The method of claim 61, wherein the antibody 11F8 mimetic is administered in combination with an anti-neoplastic agent selected from the group consisting of, a chemotherapeutic agent, irinotecan (CPT-11), and radiation.

63. The method of claim 61, wherein the antibody 11F8 mimetic is administered in combination with an agent selected from the group consisting of an EGFR antagonist an intracellular EGFR antagonist, a VEGFR antagonist, and an insulin like growth factor receptor (IGFR) antagonist.

64. A method of treating a hyperproliferative disease comprising administering a therapeutically effective amount of an antibody 11F8 mimetic of claim 59.

65. The method of claim 64, wherein the hyperproliferative disease is psoriasis.

66. The method of claim 64, wherein the antibody 11F8 mimetic is administered in combination with a topical or systemic agent for psoriasis.

67. The method of claim 64, wherein the antibody 11F8 mimetic is administered in combination with an agent selected from the list consisting of a corticosteroid and a retinoid.


SEQUENCE LISTING (8 sequences)

SEQ ID NO: 1 - protein, 7 aa, Artificial Sequence
  Chemically synthesized
SGDYYWS

SEQ ID NO: 2 - protein, 16 aa, Artificial Sequence
  Chemically synthesized
YIYYSGSTDYNPSLKS

SEQ ID NO: 3 - protein, 11 aa, Artificial Sequence
  Chemically synthesized
VSIFGVGTFDY

SEQ ID NO: 4 - protein, 11 aa, Artificial Sequence
  Chemically synthesized
RASQSVSSYLA

SEQ ID NO: 5 - protein, 7 aa, Artificial Sequence
  Chemically synthesized
DASNRAT

SEQ ID NO: 6 - protein, 9 aa, Artificial Sequence
  Chemically synthesized
HQYGSTPLT

SEQ ID NO: 7 - protein, 168 aa, Artificial Sequence
  Chemically synthesized
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKA

SEQ ID NO: 8 - protein, 168 aa, Artificial Sequence; identical to SEQ ID NO: 7
  Chemically synthesized